(19) United States
(12) Patent Application Publication
(10) Pub. No.: US 2024/0173403 A1
(43) Pub. Date: May 30, 2024

(54) Combination of AXL Antibodies and ACE Inhibitors in the Treatment of Fibrosis

(71) Applicant: BERGENBIO ASA, Bergen (NO)

(72) Inventors: Gro GAUSAL, Bergen (NO); Akil JACKSON, Bergen (NO)

(73) Assignee: BERGENBIO ASA, Bergen (NO)

(21) Appl. No.: 18/283,470

(22) PCT Filed: Mar. 23, 2022
(86) PCT No.: PCT/EP2022/057686

§ 371 Date: Sep. 22, 2023

(30) Foreign Application Priority Data
Mar. 23, 2021 (GB) 2104037.3

(51) Int. Cl. A61K 39/395; A61K 31/401; A61P 13/12

(52) U.S. Cl. CPC A61K 39/3955; A61K 31/401; A61P 13/12

(57) ABSTRACT

This disclosure relates to a combination therapy for treating subjects having a fibrotic disorder. More particularly. the disclosure relates to combination therapies comprising an AXL inhibitor (AXLi) and a renin-angiotensin system inhibitor (RASi) for treating a subject having a fibrotic disorder, as well as compositions and methods for treating subjects with said combination therapy.

### EARLIER APPLICATION

This application claims priority from United Kingdom application GB2104037.3, filed on 23 March 2021. GB2104037.3 is hereby incorporated by reference into this application in its entirety.

### FIELD

This disclosure relates to a combination therapy for treating subjects having a fibrotic disorder. More particularly, the disclosure relates to combination therapies comprising an AXL inhibitor (AXLi) and a renin-angiotensin system inhibitor (RASi) for treating a subject having a fibrotic disorder, as well as compositions and methods for treating subjects with said combination therapy.

### BACKGROUND

### AXL

All of the protein kinases that have been identified to date in the human genome share a highly conserved catalytic domain of around 300 amino acids. This domain folds into a bi-lobed structure in which resides ATP-binding and catalytic sites. The complexity of protein kinase regulation allows many potential mechanisms of inhibition including competition with activating ligands, modulation of positive and negative regulators, interference with protein dimerization, and allosteric or competitive inhibition at the substrate or ATP binding sites.

AXL (also known as UFO, ARK, and Tyro7; nucleotide accession numbers NM_021913 and NM_001699; protein accession numbers NP_068713 and NP_001690) is a receptor protein tyrosine kinase (RTK) that comprises a C-terminal extracellular ligand binding domain and N-terminal cytoplasmic region containing the catalytic domain. The extracellular domain of AXL has a unique structure that juxtaposes immunoglobulin and fibronectin Type III repeats and is reminiscent of the structure of neural cell adhesion molecules. AXL and its two close relatives, Mer/Nyk and Sky (Tyro3/Rse/Dtk), collectively known as the Tyro3 family of RTK's, all bind and are stimulated to varying degrees by the same ligand, GAS6 (growth arrest specific-6), a ˜76kDa secreted protein with significant homology to the coagulation cascade regulator, Protein S. In addition to binding to ligands, the AXL extracellular domain has been shown to undergo homophilic interactions that mediate cell aggregation, suggesting that one important function of AXL may be to mediate cell-cell adhesion.

AXL is predominantly expressed in the vasculature in both endothelial cells (EC's) and vascular smooth muscle cells (VSMC's) and in cells of the myeloid lineage and is also detected in breast epithelial cells, chondrocytes, Sertoli cells and neurons. Several functions including protection from apoptosis induced by serum starvation, TNF-α or the viral protein E1A, as well as migration and cell differentiation have been ascribed to AXL signalling in cell culture. AXL has been found to serve as a key checkpoint for interferon (IFN) signaling (Rothlin et al, 2007; Huang et al, 2015); in the context of viral responses, the Zika virus has been found to antagonize the IFN action by interacting with AXL (Chen et al, 2018). However, Axl−/− mice exhibit no overt developmental phenotype and the physiological function of AXL in vivo is not clearly established in the literature.

### AXL Pathology

The overexpression of AXL and/or its ligand has also been widely reported to have a role in the development of solid tumor types including, but not limited to, breast, renal, endometrial, ovarian, thyroid, non-small cell lung carcinoma, and uveal melanoma as well as in myeloid leukemias. The expression of AXL and its ligand GAS6 is also upregulated in a variety of other diseases including endometriosis, vascular injury, regulation of platelet responses, and—of particular interest in the present disclosure—fibrotic disorders. AXL may thus potentially represent a therapeutic target for a number of diverse pathological conditions including solid tumors (eg. breast, renal, endometrial, ovarian, thyroid, non-small cell lung carcinoma and uveal melanoma), liquid tumors (eg. leukemias—particularly myeloid leukemias—and lymphomas), endometriosis, vascular disease/injury (eg. restenosis, atherosclerosis and thrombosis), psoriasis, visual impairment (eg. macular degeneration; diabetic retinopathy, cataracts, and retinopathy of prematurity), rheumatoid arthritis, osteoporosis, osteoarthritis, and fibrotic disorders.

In solid organ fibrosis, AXL has been studied in experimental liver, lung, and renal fibrosis (Barcena et al. 2015, J. Hepatol. 63:670-678; Espindola et al. 2018 Am. J. Respir. Crit. Care Med. 197:1443-1456; Zhen et al. 2018, J. Autoimmun. 93:37-44.; Landolt et al. 20192019|Vol. 7|Iss. 10|e14091). The literature support a role for AXL in fibrosis development and an antifibrotic and anti-inflammatory role of the AXL inhibitor bemcentinib. A study on liver fibrosis showed that AXL and GAS6 are required to induce fibrogenesis by hepatic stellate cells; accordingly, exposition to bemcentinib reduced liver fibrosis in mice. In addition, less recruitment of antigen presenting cells, particularly macrophages, were detected as measured by F4/80. Less inflammation was also demonstrated by lower expression of chemokines (Barcena et al. 2015). AXL has also been seen to be up-regulated in idiopathic lung fibrosis and bemcentinib lead to a reduction in fibrosis development in two mouse lung fibrosis models (Espindola et al. 2018). Using a murine unilateral uretal obstructions (UUO) model of renal fibrosies, Landolt et al. 2019 reported that inhibition of AXL with the highl specific AXL inhibitor bemcentinib led to less fibrosis development than comparable kidneys treated with a current standard care ACE inhibitor (previously reported to produce a 52% reduction in fibrosis compared an untreated control; Moridaira et al. 2003, Am. J. Physiol. Renal Physiol. 284: F209-F217).

### AXL Inhibitors

In view of the role played by AXL in numerous pathological conditions, the development of safe and effective AXL inhibitors has been a topic of interest in recent years. Different groups of AXL inhibitors are discussed in, inter alia, US20070213375, US 20080153815, US20080188454, US20080176847, US20080188455, US20080182862, US20080188474, US20080117789,

US20090111816, WO2007/0030680, WO2008/045978, WO2008/083353, WO2008/0083357, WO2008/083354, WO2008/083356, WO2008/080134, WO2009/054864, WO/2008/083367, WO/2011/159980, WO/2016/097370, and WO/2017/220695.

### Combination Therapies Using AXL Inhibitors

The combination of one or more of the above cited AXL inhibitors with one or more other agents is discussed in, for example, WO/2010/083465 and WO/2016/193680, with WO/2016/193680 focussing on combinations of AXL inhibitors with agents having immune-regulatory or modulatory activity. For example, inhibition of AXL with the small molecule Bemcentinib (BGB324/R428) was found to enhance the efficacy of immune checkpoint inhibitor treatment with anti PD1 and/or anti CTLA4.

Landolt et al. 2019 ibid. tests the combined use of the AXL inhibitor bemcentinib with the ACE inhibitor enalapril to treat kidney fibrosis, as both agents had been separately reported to reduce kidney fibrosis when used as a monotherapy. However, the combined administration was reported not to result in any additive therapeutic effect.

The breadth and complexity of AXL biology means that research is ongoing to identify efficacious combination therapies, and specific disorders and/or subjects that will benefit most from such treatments.

### SUMMARY

The present authors sought to further investigate the role of Receptor Tyrosine Kinase AXL in fibrotic disease using Tilvestamab—a novel humanized anti-AXL antibody that blocks GAS6-mediated AXL receptor activation—in an ex vivo model of human kidney fibrosis.

In kidney fibriosis interstitial fibrosis, characterised by the accumulation of extracellular matrix in the cortical interstitium, is directly correlated with progressive chronic kidney disease secondary to inflammatory, immunologic, obstructive or metabolic causes. An invariant histologic marker of this progression is the accumulation of fibroblasts, with the phenotypic appearance of activated myofibroblasts expressing alpha smooth muscle actin (αSMA) within intracellular contractile stress fibres. Once present, these myofibroblasts are prognostic indicators of expansion of fibrotic matrix and progressive tubular atrophy, leading towards end-stage disease.

AXL is known involved in a range of kidney pathologies, with increased activity associated with Epithelial to Mesenchymal Transition (EMT) and tubular proliferation following podocyte loss. The present authors have also demonstrated enhanced expression of AXL and the mesenchymal marker, vimentin, in diseased human kidney tissue secondary to diabetes or hypertension. Futhermore, targeting AXL with the small-molecule inhibitor Bemcentinib has been reported to attenuate fibrosis and reduce inflammation in disease using the well-recognised unilateral ureteric-outflow obstruction (UUO) model of kidney fibrosis in mice (Landolt et al., 2019). The same study also tested the effect of monotherapy and combination treatments with the angiotensin-converting enzyme (ACE) inhibitor, with no additive anti-fibrotic efficacy reported for the AXLi+ACEi combination.

Consistent with the reports in Landolt et al. 2019, ibid. the present inventors found that the AXLi Bemcentinib was able to reduce production of the fibrotic markers Collagen 1a1 and TIMP-1 at all doses when delivered as a monotherapy but no such reduction was observed upon administration of Bemcentinib in combination with the ACEi Enalapril (see FIG. 1). Bemcentinib monotherapy was also observed to reduce expression of the fibrotic marker αSMA (see FIG. 2) as was Enalaril monotherapy (see FIG. 3); no benefit was observed when Bemcentinib and Enalapril were administered in combination (see FIG. 3).

Similar assays with Tilvestamab indicated that Tilvestamab monotherapy had no effect on reducing Collagen 1a1 or TIMP-1 secretion (see FIG. 1) but did reduce αSMA (see FIG. 2). However, results from the combination treatment of Tilvestamab with Enalapril were striking, with the combination having a profound effect on reducing Collagen 1a1 and TIMP-1 secretion as well as the most potent reduction of αSMA. This strong therapeutic synergy between Tilvestamab and Enalapril was both significant and unexpected.

Accordingly, in a first aspect the present disclosure provides a method of treating a fibrotic disorder, the method comprising administering to a subject in need thereof a therapeutically effective amount of an AXL inhibitor (AXLi), wherein the AXL inhibitor is administered in combination with a Renin-angiotensin system inhibitor (RASi).

The AXL inhibitor may be an antibody that binds AXL, such as human AXL. Without limiting to any particular mechanism of action, the anti-Axl antibody may antagonise Axl activity, such as inhibits Axl kinase and/or signalling activity. The anti-Axl antibody may inhibit the binding of AXL to the GAS6 ligand. The anti-Axl antibody may crosslink Axl receptors. In some cases the anti-AXL antibody comprises the CDRs of the VH domain having the sequence of SEQ ID No. 1 and/or the CDRs of VL domain having the sequence set out herein as SEQ ID No.2. In other cases the the anti-Axl antibody: comprises the CDRs of the VH domain having the sequence of SEQ ID No.33 and/or the CDRs of VL domain having the sequence set out herein as SEQ ID No.34; comprises the CDRs of the VH domain having the sequence of SEQ ID No.65 and/or the CDRs of VL domain having the sequence set out herein as SEQ ID No.66; comprises the CDRs of the VH domain having the sequence of SEQ ID No.97 and/or the CDRs of VL domain having the sequence set out herein as SEQ ID No.98; comprises the CDRs of the VH domain having the sequence of SEQ ID No. 129 and/or the CDRs of VL domain having the sequence set out herein as SEQ ID No.130; comprises the CDRs of the VH domain having the sequence of SEQ ID No. 161 and/or the CDRs of VL domain having the sequence set out herein as SEQ ID No. 162; comprises the CDRs of the VH domain having the sequence of SEQ ID No. 193 and/or the CDRs of VL domain having the sequence set out herein as SEQ ID No. 194; or comprises the CDRs of the VH domain having the sequence of SEQ ID No.225 and/or the CDRs of VL domain having the sequence set out herein as SEQ ID No.226.

In some cases the Axl inhibitor is selected from the group consisting of: bemcentinib, dubermatinib, gilteritinib, cabozantinib, SGI7079, merestinib, amuvatinib, bosutinib, glesatinib, foretinib, and TP0903.

The RAS inhibitor may be an ACE inhibitor (ACEi) such as Enalapril, Alacepril, Benazepril, Captopril, Cilazapril, Fosinopril, Imidapril, Lisinopril, Moexipril, Perindopril, Quinapril, Ramipril, Trandolapril, or Zofenopril. The RAS inhibitor may be an Angiotensin receptor blocker (ARB) such as Azilsartan, Candesartan, Eprosartan, Fimasartan, Irbesartan, Losartan, Olmesartan, Saprisartan, Telmisartan, or Valsartan. The RASi may be an aldosterone antagonists (AA) such as Spironolactone, Eplerenone, Canrenone, Finerenone, or Mexrenone.

The fibrotic disorder may be selected from the group consisting of: lung fibrosis (including pulmonary fibrosis, idiopathic pulmonary fibrosis (IPF), cystic fibrosis (CF)), cardiac fibrosis, liver fibrosis (Including non-alcoholic steatohepatitis (NASH) and primary biliary cirrhosis), kidney fibrosis, arthrofibrosis, Crohn's disease, Dupuytren's contracture, peyronie's disease, strabmisus, scleroderma, keloid, Nephrogenic systemic fibrosis, progressive massive fibrosis, fibrothorax, systemic sclerosis, cardiac fibrosis, retroperitoneal fibrosis, mediastinal fibrosis, myelofibrosis, and atherosclerosis.

In a second aspect the present disclosure provides an AXL inhibitor and a RAS inhibitor for use in a method of treating a fibrotic disorder according to the first aspect.

Also included in the second aspect are an AXL inhibitor for use in a method of treating a fibrotic disorder according to the first aspect, and a RAS inhibitor for use in a method of treating a fibrotic disorder according to the first aspect.

In a third aspect the present disclosure provides use of an AXL inhibitor and a RAS inhibitor in the manufacture of a medicament for treating a disorder in a subject, wherein the treatment comprises a method of treating a fibrotic disorder according to the first aspect.

Also included in the third aspect are use of an AXL inhibitor in the manufacture of a medicament for treating a fibrotic disorder in a subject, wherein the treatment comprises a method of treating a fibrotic disorder according to the first aspect, and the use of a RAS inhibitor in the manufacture of a medicament for treating a fibrotic disorder in a subject, wherein the treatment comprises a method of treating a fibrotic disorder according to the first aspect.

In a fourth aspect, the present disclosure provides a kit comprising an AXL inhibitor and a RAS inhibitor for use in a method of treating a fibrotic disorder according to the first aspect.

In a fifth aspect, the present disclosure provides pharmaceutical composition comprising an AXL inhibitor and/or a RAS inhibitor, and a pharmaceutically acceptable excipient, as well as such compositions for use in a method of treating a fibrotic disorder according to the first aspect.

In a sixth aspect, the present disclosure provides methods of selecting a subject to be treated in a method of treating a fibrotic disorder according to the first aspect. These include:

A method of selecting a subject for treatment with an AXL inhibitor, wherein a subject is selected for treatment if the subject has been, will be, or is being treated with a RAS inhibitor.

A method of selecting a subject for treatment with a RAS inhibitor, wherein a subject is selected for treatment if the subject has been, will be, or is being treated with an AXL inhibitor.

The disclosure includes the combination of the aspects and preferred features described except where such a combination is clearly impermissible or expressly avoided.

### SUMMARY OF THE FIGURES

FIG. 1. Collagen 1a1 levels in PCKS culture supernatants Graphs are represented by n=3 tissue culture media per condition, per time point. Data is presented as mean±SEM.

FIG. 2. % area positive tissue for α-SMA in PCKS (Bemcentinib & Tilvestamab dilution series)

Data presented as mean % area positive of tissue (±SEM) for α-SMA.

FIG. 3. % area positive tissue for α-SMA in PCKS (Bemcentinib & Tilvestamab enalapril combos)

Data presented as mean % area positive of tissue (±SEM) for α-SMA.

FIG. 4. % area positive tissue for α-SMA in PCKS

Data presented as mean % area positive of tissue (±SEM) for α-SMA.

FIG. 5.
(A) TIMP-1 levels in PCKS culture supernatants
Timp-1 was quantified in supernatants by ELISA. Data represents n=3 tissue culture media per condition, mean±SEM is shown
(B) TIMP-1 levels in PCKS culture supernatants
Timp-1 was quantified in supernatants by ELISA at 24 h (before treatment), and every 24 h until endpoint at 96 h. Graph represents AUC values (mean±SEM). Data represents n=3 tissue culture media per condition per time point.

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

The present disclosure pertains to a combination therapy for treating subjects suffering from a fibrotic disorder, and more particularly to combination therapies comprising an AXL inhibitor and a RAS inhibitor for treating subjects suffering from fibrotic disease, as well as methods of treating patients with said combination therapy.

Accordingly, the present disclosure provides a method of treating a fibrotic disorder, the method comprising administering to a subject in need thereof a therapeutically effective amount of an AXL inhibitor. In the disclosed methods of treating a fibrotic disorder, the AXL inhibitor is administered in combination with a RAS inhibitor. As used herein, “administration in combination” may mean concurrent administration or may mean separate and/or sequential administration in any order.

The present disclosure also provides an AXL inhibitor and/or a RAS inhibitor for use in a method of treating a fibrotic disorder, as well as the use of of an AXL inhibitor and/or a RAS inhibitor in the manufacture of a medicament for treating a disorder in a subject, wherein the treatment comprises a method of treating a fibrotic disorder as disclosed herein.

The present disclosure also provides methods of selecting a subject for treatment with one or more of an AXL inhibitor and/or a RAS inhibitor, as well as pharmaceutical compositions comprising an AXL inhibitor and/or a RAS inhibitor, and a pharmaceutically acceptable excipient.

### AXL Inhibitors (AXLi)

### Antibody AXL Inhibitors

In some aspects the AXL inhibitor is an antibody. In some aspects the antibody binds human AXL (see, for example, Uniprot reference P30530.1).

In some aspects, the anti-AXL antibody is an antibody as described in any of the following references: WO/2016/097370, WO/2017/220695, WO/2015/193428, WO/2016/166296, WO/2015/193430, EP2267454, WO/2009/063965, WO/2011/159980, WO/2012/175691, WO/2012/175692, WO/2013/064685, WO/2014/068139, WO/2009/062690, WO/2020/205576, and WO/2010/130751 (the contents of each of which is hereby incorporated by reference).

In another aspect, the anti-AXL antibody is an antibody as described in international patent publication WO/2020/205576, the contents of which is hereby incorporated by reference. Antibodies disclosed therein of particular interest include those comprising: (1) a VL having SEQ ID No.6 and a VH having SEQ ID NO.5, 7, 8, 9, or 10, and (2) a VL having SEQ ID No. 12 and a VH having SEQ ID NO.11.

In another aspect, the anti-AXL antibody is an antibody as described in international patent application WO/2015/193428, the contents of which is hereby incorporated by reference, particularly as shown at pages 82-83.

In another aspect, the anti-AXL antibody is an antibody as described in international patent application WO/2016/166296, the contents of which is hereby incorporated by reference, particularly the humanized 1H12 antibody disclosed therein.

In another aspect, the anti-AXL antibody is an antibody as described in international patent application WO/2015/193430, the contents of which is hereby incorporated by reference, particularly as shown at pages 72-73.

In another aspect, the anti-AXL antibody is an antibody as described in European patent publication EP2267454, the contents of which is hereby incorporated by reference.

In another aspect, the anti-AXL antibody is an antibody as described in European patent publication WO/2009/063965, the contents of which is hereby incorporated by reference, particularly as shown at pages 31-33.

In another aspect, the anti-AXL antibody is an antibody as described in US patent publication US 2012/0121587 A1, the contents of which is hereby incorporated by reference, particularly as shown at pages 26-61.

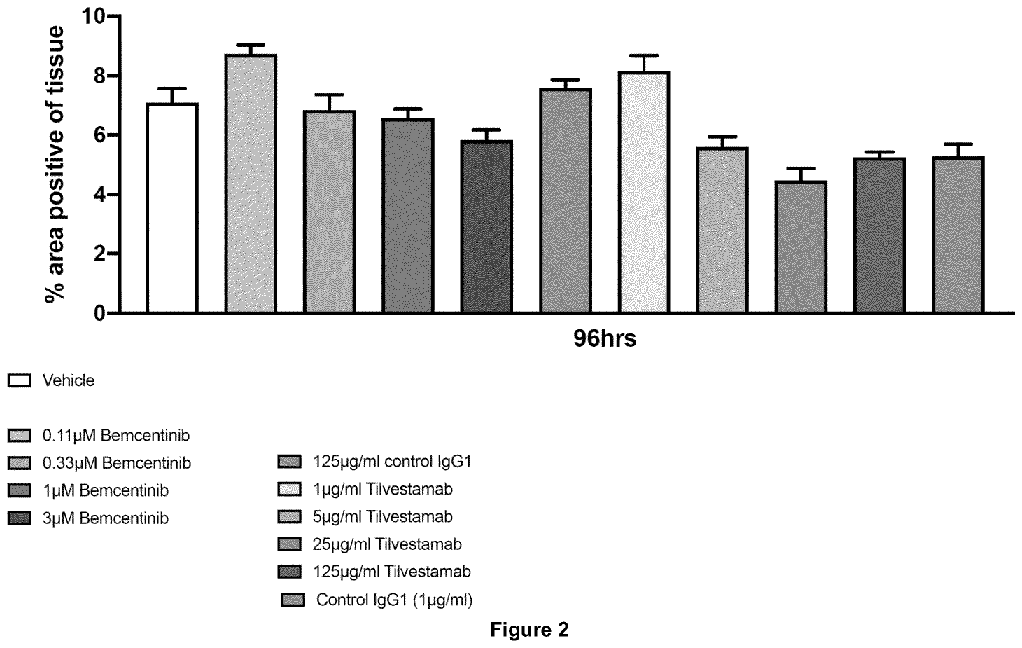

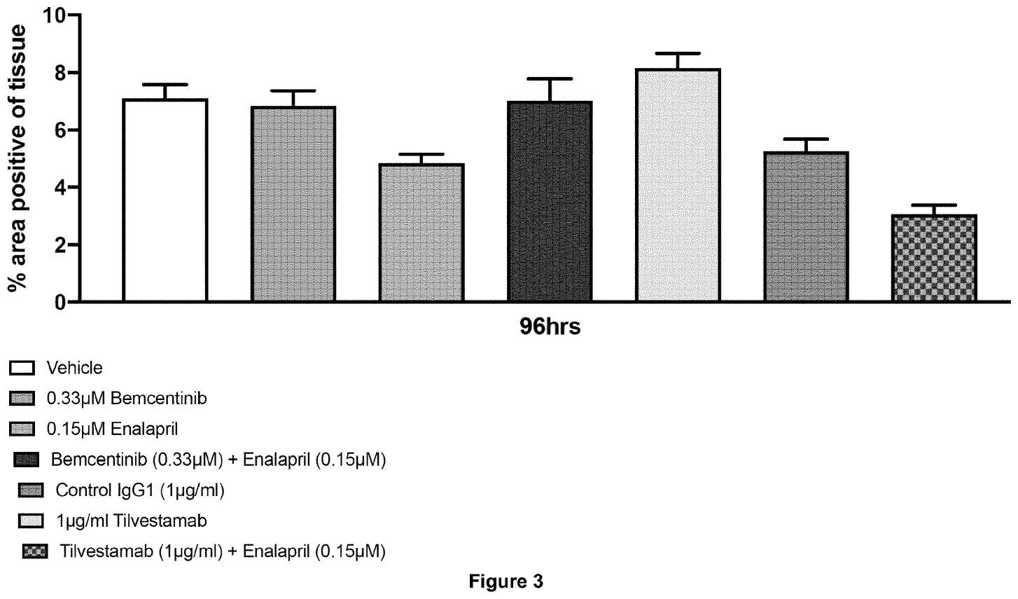

In another aspect, the anti-AXL antibody is an antibody as described in international patent publication WO/2011/159980, the contents of which is hereby incorporated by reference, particularly the YW327.6S2 antibody as shown in FIG. 2, Figure page 6 (of 24).

In another aspect, the anti-AXL antibody is an antibody as described in international patent publication WO/2012/175691, the contents of which is hereby incorporated by reference, particularly as shown at page 5.

In another aspect, the anti-AXL antibody is an antibody as described in international patent publication WO/2012/175692, the contents of which is hereby incorporated by reference, particularly as shown at pages 4-5.

In another aspect, the anti-AXL antibody is an antibody as described in international patent publication WO/2009/062690, the contents of which is hereby incorporated by reference.

In another aspect, the anti-AXL antibody is an antibody as described in international patent publication WO/2010/130751, the contents of which is hereby incorporated by reference, particularly as shown at pages 1-17 (of 78).

In another aspect, the anti-AXL antibody is an antibody as described in international patent publication WO/2013/064685, the contents of which is hereby incorporated by reference, particularly the 1613F12 antibody described therein as shown at, for example, Examples 6 to 8.

In another aspect, the anti-AXL antibody is an antibody as described in international patent publication WO/2014/068139, the contents of which is hereby incorporated by reference, particularly the 110D7, 1003A2, and 1024G11 antibodies described therein as shown at, for example, Examples 6 to 8.

In another aspect, the anti-AXL antibody is an antibody as described in international patent publication WO/2016/097370, the contents of which is hereby incorporated by reference, particularly the murine 10G5 and 10C9 antibodies described therein as shown at, for example, Examples 6 to 8.

In another aspect, the anti-AXL antibody is an antibody as described in international patent publication WO/2017/220695, the contents of which is hereby incorporated by reference, particularly the humanized 10G5 antibody described therein as shown at, for example, SEQ ID NO. 1 to 10.

Antibodies that Antagonise Axl Activity

Without wishing to limit to any particular mechansms of action, in some aspects the antibody antagonises Axl activity, such as Axl kinase and/or signalling activity (termed herein an “antagonistic anti-Axl antibody”).

In some cases the antibody inhibits the binding of AXL to the GAS6 ligand. In some cases the inhibition of GAS6 binding is measured using the competitive binding assay described in Example 6 of WO2017/220695 which is herein incorporated by reference. In some cases the inhibition of GAS6 binding is measured as follows:
A competitive binding study using Biacore 3000 instrument (GE Healthcare) and Binding Analysis wizard with several cycles of two samples injection. As a first sample, a saturating concentration of antibody (Ab) (160 nM or 24 μg/mL) is injected over the surface of a CM5 sensor chip coated with rhAxl-Fc (using amine coupling) for 3 min at flow rate of 30 μL/min followed by 2.5 min stabilization (HBS-EP buffer alone) before the injection of the second sample. The following second samples are used: recombinant human (rh) Gas6 (R&D Systems, Cat. no. 885-GS), recombinant mouse (rm) Gas6 (R&D Systems, Cat. no. 986-GS/CF) and a panel of control anti-Axl antibodies; all at concentration 25 μg/mL. The second sample is injected for 3 min, followed by 2.5 min stabilization (buffer alone) and regeneration of the surface by 30 sec injection of a regeneration solution (10 mM HCl, 1 M NaCl) at flow rate 50 μL/min.

Again, without wishing to limit to any particular mechansms of action, in some aspects the antibody is able to crosslink and/or cluster Axl receptors. In some cases the antibody is bivalent, trivalent, tetravalent, or higher-order multivalent.

Antibodies having anatagoistic activity include:
(1) Tilvestamab;
(2) the humanized 10G5 antibody described in WO/2017/220695;
(3) the murine 10G5 antibody described in WO/2016/097370;
(4) the murine 10C9 antibody described in WO/2016/097370;
(5) the YW327.6S2 antibody described in WO/2011/159980;
(6) the 1003A2 antibody described in WO/2014/068139;
(7) the 1613F12 antibody described in WO/2013/064685;
(8) the 110D7 antibody described in WO/2014/068139; and
(9) the 1024G11 antibody described in WO/2014/068139.

Accordingly, in some aspects the anti-AXL antibody comprises the CDRs of the VH domain having the sequence set out herein as SEQ ID No.1 and/or the CDRs of VL domain having the sequence set out herein as SEQ ID No.2. The anti-AXL antibody may comprise all 6 of the CDRs present in the VH and VL sequences.

In some aspects the anti-AXL antibody comprises the 6 CDRs having the sequences set out herein as: SEQ ID Nos. 3 to 8, SEQ ID Nos. 9 to 14, SEQ ID Nos. 15 to 20, SEQ ID Nos. 21 to 26, or SEQ ID Nos. 27 to 32.

In some aspects the anti-AXL antibody comprises a VH domain having the sequence set out herein as SEQ ID No.1 and/or the VL domain having the sequence set out herein as SEQ ID No.2.

In some aspects the anti-AXL antibody is Tilvestamab.

In some aspects the anti-AXL antibody comprises the CDRs of the VH domain having the sequence set out herein as SEQ ID No.33 and/or the CDRs of the VL domain having the sequence set out herein as SEQ ID No.34. The anti-AXL antibody may comprise all 6 of the CDRs present in the VH and VL sequences.

In some aspects the anti-AXL antibody comprises the 6 CDRs having the sequences set out herein as: SEQ ID Nos. 35 to 40, SEQ ID Nos. 41 to 46, SEQ ID Nos. 47 to 52, SEQ ID Nos. 53 to 58, or SEQ ID Nos. 59 to 64.

In some aspects the anti-AXL antibody comprises a VH domain having the sequence set out herein as SEQ ID No.33 and/or the VL domain having the sequence set out herein as SEQ ID No.34.

In some aspects the anti-AXL antibody comprises the CDRs of the VH domain having the sequence set out herein as SEQ ID No.65 and/or the CDRs of the VL domain having the sequence set out herein as SEQ ID No.66. The anti-AXL antibody may comprise all 6 of the CDRs present in the VH and VL sequences.

In some aspects the anti-AXL antibody comprises the 6 CDRs having the sequences set out herein as: SEQ ID Nos. 67 to 72, SEQ ID Nos. 73 to 78, SEQ ID Nos. 79 to 84, SEQ ID Nos. 85 to 90, or SEQ ID Nos. 91 to 96.

In some aspects the anti-AXL antibody comprises a VH domain having the sequence set out herein as SEQ ID No.65 and/or the VL domain having the sequence set out herein as SEQ ID No.66.

In some aspects the anti-AXL antibody comprises the CDRs of the VH domain having the sequence set out herein as SEQ ID No.97 and/or the CDRs of the VL domain having the sequence set out herein as SEQ ID No.98. The anti-AXL antibody may comprise all 6 of the CDRs present in the VH and VL sequences.

In some aspects the anti-AXL antibody comprises the 6 CDRs having the sequences set out herein as: SEQ ID Nos. 99 to 104, SEQ ID Nos. 105 to 110, SEQ ID Nos. 111 to 116, SEQ ID Nos. 117 to 122, or SEQ ID Nos. 123 to 128.

In some aspects the anti-AXL antibody comprises a VH domain having the sequence set out herein as SEQ ID No.97 and/or the VL domain having the sequence set out herein as SEQ ID No.98.

In some aspects the anti-AXL antibody comprises the CDRs of the VH domain having the sequence set out herein as SEQ ID No. 129 and/or the CDRs of the VL domain having the sequence set out herein as SEQ ID No.130. The anti-AXL antibody may comprise all 6 of the CDRs present in the VH and VL sequences.

In some aspects the anti-AXL antibody comprises the 6 CDRs having the sequences set out herein as: SEQ ID Nos. 131 to 136, SEQ ID Nos. 137 to 142, SEQ ID Nos. 143 to 148, SEQ ID Nos. 149 to 154, or SEQ ID Nos. 155 to 160.

In some aspects the anti-AXL antibody comprises a VH domain having the sequence set out herein as SEQ ID No. 129 and/or the VL domain having the sequence set out herein as SEQ ID No.130.

In some aspects the anti-AXL antibody comprises the CDRs of the VH domain having the sequence set out herein as SEQ ID No. 161 and/or the CDRs of the VL domain having the sequence set out herein as SEQ ID No.162. The anti-AXL antibody may comprise all 6 of the CDRs present in the VH and VL sequences.

In some aspects the anti-AXL antibody comprises the 6 CDRs having the sequences set out herein as: SEQ ID Nos. 163 to 168, SEQ ID Nos. 169 to 174, SEQ ID Nos. 175 to 180, SEQ ID Nos. 181 to 186, or SEQ ID Nos. 187 to 192.

In some aspects the anti-AXL antibody comprises a VH domain having the sequence set out herein as SEQ ID No. 161 and/or the VL domain having the sequence set out herein as SEQ ID No.162.

In some aspects the anti-AXL antibody comprises the CDRs of the VH domain having the sequence set out herein as SEQ ID No. 193 and/or the CDRs of the VL domain having the sequence set out herein as SEQ ID No. 194. The anti-AXL antibody may comprise all 6 of the CDRs present in the VH and VL sequences.

In some aspects the anti-AXL antibody comprises the 6 CDRs having the sequences set out herein as: SEQ ID Nos. 195 to 200, SEQ ID Nos. 201 to 206, SEQ ID Nos. 207 to 212, SEQ ID Nos. 213 to 218, or SEQ ID Nos. 219 to 224.

In some aspects the anti-AXL antibody comprises a VH domain having the sequence set out herein as SEQ ID No. 193 and/or the VL domain having the sequence set out herein as SEQ ID No. 194.

In some aspects the anti-AXL antibody comprises the CDRs of the VH domain having the sequence set out herein as SEQ ID No.225 and/or the CDRs of the VL domain having the sequence set out herein as SEQ ID No.226. The anti-AXL antibody may comprise all 6 of the CDRs present in the VH and VL sequences.

In some aspects the anti-AXL antibody comprises the 6 CDRs having the sequences set out herein as: SEQ ID Nos. 227 to 232, SEQ ID Nos. 233 to 238, SEQ ID Nos. 239 to 244, SEQ ID Nos. 245 to 250, or SEQ ID Nos. 251 to 256.

In some aspects the anti-AXL antibody comprises a VH domain having the sequence set out herein as SEQ ID No.225 and/or the VL domain having the sequence set out herein as SEQ ID No.226.

### Small Molecule AXL Inhibitors

In some cases the AXLi is a small molecule inhibitor.

The AXL inhibitor may be 1-(6,7-dihydro-5H-benzo[6,7]cyclohepta[1,2-c]pyridazin-3-yl)-N³-((7-(S)-pyrrolidin-1-yl)-6,7,8,9-tetrahydro-5H-benzo[7]annulene-2-yl)-1H-1,2,4-triazole-3,5-diamine. The AXL inhibitor may be bemcentinib (CAS No. 1037624-75-1; UNII 0ICW2LX8AS). Bemcentinib may be referred to as BGB324 or R428.

### Other Small Molecule Aspects

In some other aspects the AXL inhibitor is selected from the group consisting of:
Dubermatinib (CAS No. 1341200-45-0; UNII 14D65TV20J);
Gilteritinib (CAS No. 1254053-43-4; UNII 66D92MGC8M);
Cabozantinib (CAS No. 849217-68-1; UNII 1C39JW444G);
SGI7079 (CAS No. 1239875-86-5);
Merestinib (CAS No. 1206799-15-6; UNII 50GS5K699E);
Amuvatinib (CAS No. 850879-09-3; UNII SO9S6QZB4R);
Bosutinib (CAS No. 380843-75-4; UNII 5018V4AEZ0);
Glesatinib (CAS No. 936694-12-1; UNII 7Q290XD98N); and
foretinib (CAS No. 849217-64-7; UNII 81FH7VK1C4).
TP0903 (CAS No. 1341200-45-0).

In some other aspects the AXL inhibitor is an AXL inhibitor as described in any of the following references: WO2008/083367, WO2010/083465, and WO2012/028332 (the contents of each of which is hereby incorporated by reference).

### Renin-Angiotensin System Inhibitors (RASi)

The renin-angiotensin system (RAS) is well known for its regulation of blood pressure and fluid homeostasis. However, it is also involved in organ dysfunction and chronic tissue damage, via the vasoactive and profibrotic effects of angiotensin (Ang) II, a major effector octapeptide (Kwang et al., Korean J Intern Med. 2018 May; 33(3): 453-461.).

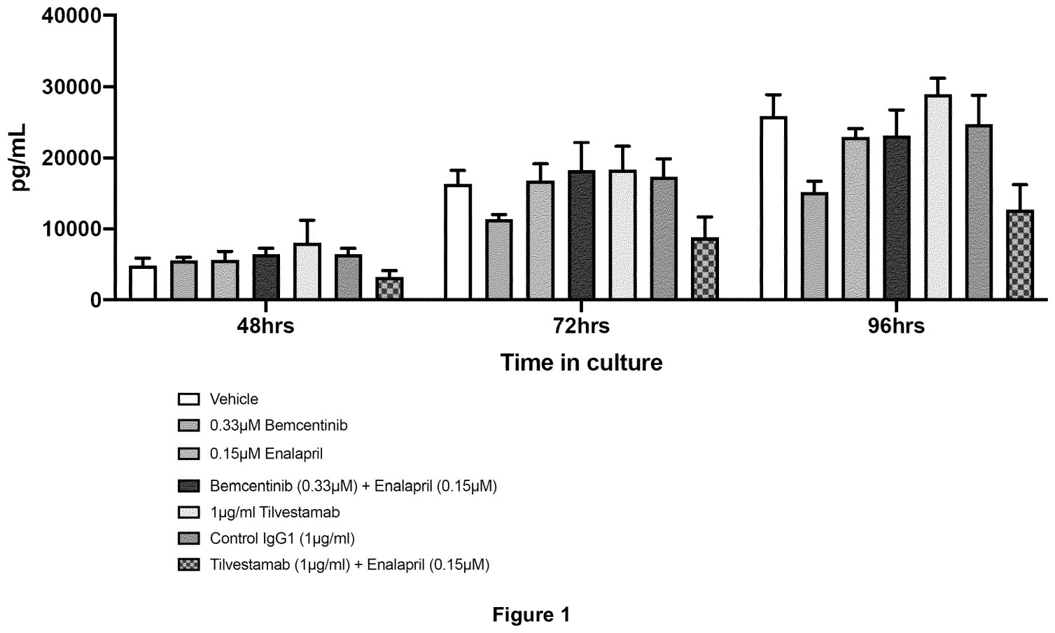

Angiotensin II (Ang-II), the final effector of the system, causes vasoconstriction both directly and indirectly by stimulating Ang-Il type 1 receptor (AT-1) receptors present on the vasculature and by increasing sympathetic tone and arginine vasopressin release. Chronically, Ang-Il regulates blood pressure by modulating renal sodium and water reabsorption directly, by stimulating AT-1 receptors in the kidney, or indirectly, by stimulating the production and release of aldosterone from the adrenal glands, or stimulating the sensation of thirst in the central nervous system (CNS). The enzymatic cascade by which Ang-Il is produced consists of renin (REN), an aspartyl protease, which cleaves angiotensinogen (AGT) to form the decapeptide angiotensin I (Ang-I; FIG. 1). Ang-I is then further cleaved by angiotensin-converting enzyme (ACE), a dipeptidyl carboxypeptidase, to produce the octapeptide Ang-II, the physiologically active component of the system. Further degradation (or processing) by aminopeptidase A and N produces angiotensin III (Ang 2-8), and angiotensin IV (Ang 3-8), respectively. The actions of Ang-Il results from its binding to specific receptors (AT-1 and AT-2), classified by their differential affinities for various nonpeptide antagonists (1). Both of these cell surface receptors belong to the large family of G protein-coupled receptors although the pathways used are completely different and signal in apparent opposition. For example, AT-1 receptors mediate vasoconstrictor responses whereas AT-2 receptors are thought to mediate vasodilator responses. The AT-1 and AT-2 receptors have a wide tissue-specific distribution, and are both present in the kidney, brain, and adrenal gland.

RAS activity is anatgonised by several classes of compounds, including ACE inhibitors (ACEis), Angiotensin receptor blockers (ARBs), and aldosterone antagonists (AAs).

ACE inhibitors

| | CAS registry | Unique Ingredient |
|---|---|---|
| | number\hat{ } | Identifier (UNII)* |
| Alacepril | 74258-86-9 | X39TL7JDPF |
| Enalapril | 75847-73-3 | 69PN84IO1A |
| Benazepril | 86541-75-5 | UDM7Q7QWP8 |
| Captopril | 62571-86-2 | 9G64RSX1XD |
| Cilazapril | 88768-40-5 | 8Q9454114Q |
| Fosinopril | 98048-97-6 | R43D2573WO |
| Imidapril | 89371-37-9 | BW7H1TJS22 |
| Lisinopril | 83915-83-7 | E7199S1YWR |
| Moexipril | 103775-10-6 | WT87C52TJZ |
| Perindopril | 82834-16-0 | Y5GMK36KGY |
| Quinapril | 85441-61-8 | RJ84Y44811 |
| Ramipril | 87333-19-5 | L35JN3I7SJ |
| Trandolapril | 87679-37-6 | 1T0N3G9CRC |
| Zofenopril | 81872-10-8 | 290ZY759PI |

\hat{ }https://www.cas.org/services/content/registry-lookup, https://www.commonchemistry.org/
*https://fdasis.nlm.nih.gov/srs/

ARBs

| | CAS registry | Unique Ingredient |
|---|---|---|
| | number\hat{ } | Identifier (UNII)* |
| Azilsartan | 147403-03-0 | F9NUX55P23 |
| Candesartan | 139481-59-7 | S8Q36MD2XX |
| Eprosartan | 133040-01-4 | 2KH13Z0S0Y |
| Fimasartan | 247257-48-3 | P58222188P |
| Irbesartan | 138402-11-6 | J0E2756Z7N |
| Losartan | 114798-26-4 | JMS50MPO89 |
| Olmesartan | 144689-63-4 | 6M97XTV3HD |
| Saprisartan | 146623-69-0 | HS64NG1G69 |
| Telmisartan | 144701-48-4 | U5SYW473RQ |
| Valsartan | 137862-53-4 | 80M03YXJ7I |

\hat{ }https://www.cas.org/services/content/registry-lookup, https://www.commonchemistry.org/
*https://fdasis.nlm.nih.gov/srs/

AAs

| | CAS registry | Unique Ingredient |
|---|---|---|
| | number\hat{ } | Identifier (UNII)* |
| Spironolactone | 52-01-7 | 27O7W4T232 |
| Eplerenone | 107724-20-9 | 6995V82D0B |
| Canrenone | 976-71-6 | 78O20X9J0U |
| Finerenone | 1050477-31-0 | DE2O63YV8R |
| Mexrenone | 41020-65-9 | 16FTD41OAV |

\hat{ }https://www.cas.org/services/content/registry-lookup, https://www.commonchemistry.org/
*https://fdasis.nlm.nih.gov/srs/

In some aspects the RAS inhibitor is any agent which inhibits RAS activity. For example, the RASi may be any agent which reduces RAS activity by at least 10%, such as at least 20%, at least 30%, at least 40%, at least 50%, at least 60%, at least 70%, at least 80%, at least 90%, at least 95%, or at least 98%.

In some aspects the RASi is an ACE inhibitors (ACEi). In some aspects the ACEi is any agent which inhibits ACE enzymatic activity. For example, the ACEi may be any agent which reduces ACE activity by at least 10%, such as at least 20%, at least 30%, at least 40%, at least 50%, at least 60%, at least 70%, at least 80%, at least 90%, at least 95%, or at least 98%. In some aspects the ACEi activity is measured using the Elbl and Wagner assay (Elbl G, Wagner H., Planta Med. 1991;57:137-41). In some aspects the ACEi is selected from the group consisting of Enalapril, Alacepril, Benazepril, Captopril, Cilazapril, Fosinopril, Imidapril, Lisinopril, Moexipril, Perindopril, Quinapril, Ramipril, Trandolapril, and Zofenopril.

In some aspects the RASi is an Angiotensin receptor blocker (ARB). In some aspects the ARBi is any agent which inhibits Angiotensin receptor binding. For example, the ARB may be any agent which reduces Angiotensin receptor binding by at least 10%, such as at least 20%, at least 30%, at least 40%, at least 50%, at least 60%, at least 70%, at least 80%, at least 90%, at least 95%, or at least 98%. In some aspects Angiotensin receptor binding is measured using the receptor binding assay described in Maillard, MP., et al., American Journal of Hypertension, Volume 12, Issue 12, Dec. 1999, Pages 1201-1208. In some aspects the ARB is selected from the group consisting of Azilsartan, Candesartan, Eprosartan, Fimasartan, Irbesartan, Losartan, Olmesartan, Saprisartan, Telmisartan, and Valsartan.

In some aspects the RASi is an aldosterone antagonists (AA). In some aspects the AA is any agent which inhibits binding to the Mineralcorticoid receptor. For example, the ARB may be any agent which reduces mineralcorticoid receptor binding (as—for example—assessed by the constant of inhibition, Ki) by at least 10%, such as at least 20%, at least 30%, at least 40%, at least 50%, at least 60%, at least 70%, at least 80%, at least 90%, at least 95%, or at least 98%. In some aspects mineralcorticoid receptor binding is measured using the receptor binding assay described in Fujihara, CK., et al., Scientific Reports volume 7, Article number: 7899 (2017). In some aspects the AA is selected from the group consisting of Spironolactone, Eplerenone, Canrenone, Finerenone, and Mexrenone.

In some aspects of the disclosed methods, the RASi is an ACEi. In some aspects the ACEi is Enalapril.

### Fibrotic Disorders

As referred to herein, the terms “fibrotic disorder” and “fibrosis” are used interchangeably to refer to the excessive accumulation of extracellular matrix (ECM) that leading to distortion of tissue architecture and often pathological loss of organ function (Jun, JI., J Clin Invest. 2018; 128(1):97-107). This pathology commonly results from a wound healing response to repeated or chronic injury or tissue damage, irrespective of the underlying etiology, and can occur in virtually any solid organ or tissue. A broad range of prevalent chronic diseases can give rise to fibrosis, including diabetes, hypertension, viral and nonviral hepatitis, heart failure and cardiomyopathy, idiopathic pulmonary disease, scleroderma, and cancer. Fibrosis resulting from these and other diseases can lead to failure of liver, lung, kidney, heart, or other vital organs as excessive ECM replaces and disrupts parenchymal tissue (Rockey DC et al., N Engl J Med. 2015; 372(12):1138-1149). Consequently, severe fibrosis is estimated to account for up to 45% of all deaths in the developed world (Wynn TA., Nat Rev Immunol. 2004; 4(8):583-594.).

Wound healing in any organ generally proceeds through three broad phases that are temporally overlapping but functionally distinct. Immediately following injury, hemostasis is achieved through the formation of a platelet plug and a fibrin matrix, accompanied by the release of cytokines and chemokines that initiate inflammation and recruit immune cells. This leads to the first phase of healing, the inflammatory phase, wherein infiltration of neutrophils and macrophages combats possible infections and removes tissue and cell debris. Cells undergoing apoptosis and the immune cells they recruit promote new tissue formation by producing proinflammatory, vasoactive, and profibrotic effectors, including TGF-β1, PDGF, TNF-α, IL-6, and IL-13, to prompt the proliferative phase of healing. TGF-β1 plays a particularly prominent role in inducing the differentiation of precursor cells into myofibroblasts, which rapidly produce a prodigious amount of ECM to maintain the integrity of the injured tissue during repair and to enhance cell proliferation for granulation tissue formation or parenchymal regeneration. In the final maturation phase, the provisional ECM is degraded and remodeled to rebuild the parenchymal tissue architecture. Dysregulation of these processes or repeated or chronic injury allows inadequate opportunity for the ECM to be resolved as myofibroblasts are relentlessly stimulated to produce ECM. Over time, the accumulated ECM begins to form a fibrotic lesion. With some exceptions, fibrosis is associated with chronic inflammation, which drives the production of profibrotic growth factors (Stramer BM, et al., J Invest Dermatol. 2007; 127(5):1009-1017).

The principal cell type that produces ECM to form fibrotic lesions is the myofibroblast, which exhibits features of both smooth muscle cells and fibroblasts, and is characterized by a prominent rough endoplasmic reticulum, stress fibers, enlarged nucleolus, and expression of α-smooth muscle actin (αSMA) and other contractile proteins (Bochaton-Piallat ML et al., F1000Res. 2016;5:F1000 Faculty Rev-752). Myofibroblasts produce interstitial or fibrillar ECM largely composed of collagen I and III, as well as myriad other ECM proteins, including an alternatively spliced form of fibronectin with an extra domain A (EDA-fibronectin) that is important for TGF-B1-induced myofibroblast differentiation. In the early granulation tissue, there is a population of fibroblastic cells that express collagen I and III, termed proto-myofibroblasts, that can be fully differentiated into myofibroblasts upon stimulation by TGF-β1. The sources of myofibroblast precursors are heterogeneous and have been controversial, although recent data indicate that resident fibroblasts and pericytes are major contributors, with mesenchymal stem cells also playing a role.

In some aspects of the disclosure, the fibrotic disorder is selected from the group consisting of arthrofibrosis, Crohn's disease, Dupuytren's contracture, peyronie's disease, strabmisus, scleroderma, keloid, Nephrogenic systemic fibrosis, pulmonary fibrosis, idiopathic pulmonary fibrosis (IPF), cystic fibrosis (CF), progressive massive fibrosis, fibrothorax, systemic sclerosis, cardiac fibrosis, non-alcoholic steatohepatitis (NASH), other types of liver fibrosis, primary biliary cirrhosis, renal fibrosis, reteroperitoneal fibrosis, mediastinal fibrosis, myelofibrosis, and atherosclerosis. In these diseases, the chronic development of fibrosis in tissue leads to marked alterations in the architecture of the affected organs and subsequently cause defective organ function.

Four fibrotic disorders of particular interest are Liver (hepatic) fibrosis, Lung (pulmonary) fibrosis, Kidney (renal) fibrosis, and Heart (cardiac) fibrosis. These are therefore described below in more detail.

Liver fibrosis. The primary causes of liver fibrosis are etiologies that drive chronic inflammation, including viral and parasitic infections, excessive alcohol consumption, and nonalcoholic steatohepatitis. The most compelling evidence for the resolution of organ fibrosis in humans is observed in the liver. Patients with liver fibrosis associated with hepatitis B virus (HBV) or HCV infection treated with antiviral therapies have shown fibrosis regression and histological improvements even in cases of cirrhosis (Bachofner JA, et al., Liver Int. 2017; 37(3):369-376.). Common experimental rodent models for liver fibrosis include administration of a hepatotoxin (e.g., carbon tetrachloride [CCl₄]) to induce acute hepatocellular injury or bile duct ligation (BDL) to induce cholestasis, resulting in pericentral or periportal liver fibrosis, respectively. Genetic lineage tracing studies have found that the vast majority of myofibroblasts in the fibrotic rodent liver are derived from hepatic stellate cells (HSCs), irrespective of etiology (Iwaisako K, et al., Proc Natl Acad Sci U S A. 2014; 111(32):E3297-E3305.). HSCs are normally quiescent, pericyte-like vitamin A storage cells located in the space of Disse between hepatocytes and the fenestrated sinusoid. In addition, portal fibroblasts, bone marrow-derived fibrocytes, and Gli+ mesenchymal stem cell-like cells also contribute to hepatic myofibroblasts (Iwaisako K, et al., ibid.), whereas mesothelial cells can give rise to HSCs and myofibroblasts through mesothelial-to-mesenchymal transition (MMT) (Li Y, et al., PNAS USA. 2013; 110(6):2324-2329).

Lung fibrosis. Fibrosis of the lung is associated with diverse etiologies, including scleroderma (systemic sclerosis), sarcoidosis, infections, and exposure to toxicants or radiation. Idiopathic pulmonary fibrosis (IPF) is the most common form of idiopathic interstitial pneumonia and is usually fatal, with a median survival of 2 to 3 years. In 2014, the FDA granted fast-track approval for the profibrotic signaling inhibitors pirfenidone and nintedanib for treating IPF on the basis of their slowing of lung function decline as measured by forced vital capacity and reduced all-cause mortality (Karimi-Shah BA,, N Engl J Med. 2015; 372(13): 1189-1191). However, the efficacy of these drugs to promote fibrosis resolution in IPF has not been demonstrated. The most common experimental mouse model for lung fibrosis is intratracheal administration of the chemotherapeutic drug bleomycin, which induces inflammation followed by fibrosis. Spontaneous resolution of fibrosis with restitution of tissue architecture occurs in this model upon cessation of further bleomycin-induced injury.

Kidney fibrosis. Fibrosis is a major complication in all forms of chronic kidney disease (CKD), of which diabetes and hypertension are principal causes. Even though the initial injury to the kidney may affect the glomerulus, tubules, or interstitium, the final outcome of all progressive CKD is the formation of tubulointerstitial fibrosis (Liu Y., Nat Rev Nephrol. 2011; 7(12):684-696). Kidney fibrosis in patients with diabetic nephropathy can be ameliorated by pancreas transplantation, suggesting that established kidney fibrosis can be reversible to some extent (Duffield JS., J Clin Invest. 2014; 124(6):2299-2306.). The common rodent model of renal fibrosis unilateral ureteral obstruction (UUO) causes fibrosis to develop within 7 days (Landolt et al. 2019, ibid.). Interestingly, recent studies have indicated that tubular epithelial cells can undergo partial epithelial-to-mesenchymal transition, leading to recruitment of inflammatory cells and release of fibrogenic cytokines, which exacerbate myofibroblast activation and fibrogenesis (Lovisa S, et al., Nat Med. 2015; 21(9):998-1009).

Cardiac fibrosis. Cardiac fibrosis results from pathological myocardial remodeling triggered by heart diseases of nearly all etiologies (Travers JG, Circ Res. 2016; 118(6): 1021-1040). In contrast to organs discussed above, the parenchymal cells in the heart are muscle cells (cardiomyocytes) rather than epithelial cells and display very limited regenerative capacity. Consequently, extensive scarring is necessary to prevent rupture following myocardial infarction and other injuries. Nevertheless, in patients with hypertension and left ventricular hypertrophy or stiffness, regression of biopsy-proven cardiac fibrosis was observed after treatment with the hypotensive drugs lisinopril or losartan (Brilla CG,, Circulation. 2000;102(12):1388-1393; Diez J, Circulation. 2002;105(21):2512-2517). Experimental models of cardiac fibrosis include permanent occlusion of the left anterior descending (LAD) coronary artery to induce myocardial infarction, and transverse aortic constriction (TAC) to trigger pressure overload-induced cardiac hypertrophy. Regression of collagen deposition occurs after constriction is removed in TAC-treated mice and in hypertensive rats following treatment with angiotensin-converting enzyme inhibitors (Tyralla K, et al., PLOS One. 2011; 6(1):e15287.).

Accordingly, in some aspects the fibrotic disorder is selected from the group consisting of lung fibrosis (including pulmonary fibrosis, idiopathic pulmonary fibrosis (IPF), cystic fibrosis (CF)), cardiac fibrosis, liver fibrosis (Including non-alcoholic steatohepatitis (NASH) and primary biliary cirrhosis), and kidney fibrosis.

In some aspects the fibrotic disorder is kidney fibrosis.

### Pattern of Administration

Accordingly, as used herein, “administration in combination” may mean concurrent administration or may mean separate and/or sequential administration in any order. Thus, in some aspects of the disclosure, the AXL inhibitor and RAS inhibitor may be administered concurrently. In other aspects the AXL inhibitor and RAS inhibitor may be administered separately and/or sequentially. In other aspects the AXL inhibitor and RAS inhibitor may be administered separately and/or sequentially. In some aspects, the AXL inhibitor may be administered subsequent to administration of the RAS inhibitor. In some aspects, the AXL inhibitor may be administered before the administration of the RAS inhibitor.

In some aspects of the disclosed methods of treating a fibrotic disorder, the method comprises: administering the AXL inhibitor to the subject, when the RAS inhibitor has been, is, or will be, administered to the subject.

In some aspects of the disclosed methods of treating a fibrotic disorder, the method comprises: administering the RAS inhibitor to the subject, when the AXL inhibitor has been, is, or will be, administered to the subject.

In aspects where the AXL inhibitor and RAS inhibitor are not administered concurrently, in some aspects the AXL inhibitor and ICM are administered to the subject no more than 1 week apart, such as no more than 48 hours apart, no more than 24 hours apart, no more than 12 hours apart, no more than 6 hours apart, no more than 2 hours apart, or no more than 1 hour apart.

### Methods of Treatment

As outlined above, the present disclosure provides a method of treating a fibrotic disorder, the method comprising administering to a subject in need thereof a therapeutically effective amount of an AXL inhibitor. In the disclosed methods of treating a fibrotic disorder, the AXL inhibitor may be administered in combination with a RAS inhibitor. As used herein, “administration in combination” may mean concurrent administration or may mean separate and/or sequential administration in any order.

Thus, the present disclosure provides a method of treating a fibrotic disorder, the method comprising administering to a subject in need thereof a therapeutically effective amount of an AXL inhibitor, wherein the AXL inhibitor is administered in combination with a RAS inhibitor.

Also provided are methods of treating a fibrotic disorder, the method comprising administering to a subject in need thereof a therapeutically effective amount of an AXL inhibitor, wherein the subject has been or will be administered a RAS inhibitor.

In some aspects of the methods of the disclosure, the AXL inhibitor and RAS inhibitor are administered to the subject no more than 4 weeks apart, such as no more than 3 weeks, no more than 1 week apart, no more than 48 hours apart, or no more than 24 hours apart. That is, in some aspects the AXL inhibitor may be administered to the subject within 4 weeks, within 3 weeks, within 1 week, of the RAS inhibitor being administered to the subject. For example, in some aspects the AXL inhibitor may be administered to the subject 4 weeks, 3 weeks, or 1 week after administration of the RAS inhibitor. In other aspects, the the AXL inhibitor may be administered to the subject 4 weeks, 3 weeks, or 1 week before administration of the RAS inhibitor.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

Typically, in the methods of treatment described herein the agents (AXL inhibitors, RAS inhibitors) are administered in a therapeutically or prophylactically effective amount. The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Typically, the subjects treated are in need of the described treatment.

A “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

The disclosed methods of treatment may involve administration of the AXLi plus RASi combination of the disclosure alone or in further combination with other treatments, either simultaneously or sequentially dependent upon the condition to be treated. Examples of treatments and therapies include, but are not limited to, chemotherapy (the administration of active agents, including, e.g. drugs, such as chemotherapeutics) and surgery.

### Compositions, Uses, and Kits

In addition to methods of treating a fibrotic disorder, the present disclosure provides compositions comprising an AXL inhibitor and/or RAS inhibitor, as well as the use of such compositions in the disclosed methods of treating a fibrotic disease.

Accordingly, the present disclosure provides an AXL inhibitor and a RAS inhibitor for use in a method of treatment according to the present disclosure. Also provided is an AXL inhibitor for use in a method of treatment according to the present disclosure, or a RAS inhibitor for use in a method of treatment according to the present disclosure.

Thus, the present disclosure provides an AXL inhibitor and a RAS inhibitor for use in a method of treating a fibrotic disorder. Also provided is an AXL inhibitor for use in a method of treating a fibrotic disorder, the method comprising administering to a subject in need thereof a therapeutically effective amount of an AXL inhibitor, wherein the AXL inhibitor is administered in combination with a RAS inhibitor. Also provided is a RAS inhibitor for use in a method of treating a fibrotic disorder, the method comprising administering to a subject in need thereof a therapeutically effective amount of an RAS inhibitor, wherein the RAS inhibitor is administered in combination with: an AXL inhibitor.

Also provided is the use of an AXL inhibitor and a RAS inhibitor in the manufacture of a medicament for treating a fibrotic disorder in a subject, wherein the treatment comprises a method of treatment according to the present disclosure. Also provided is the use of an Axl inhibitor in the manufacture of a medicament for treating a fibrotic disorder in a subject, wherein the treatment comprises a method of treatment according to the present disclosure; and the use of a RAS inhibitor in the manufacture of a medicament for treating a disorder in a subject, wherein the treatment comprises a method of treatment according to the present disclosure.

The present disclosure also provides a kit comprising an AXL inhibitor and a RAS inhibitor for use in a method of treating a fibrotic disorder as disclosed herein.

Compositions according to the present disclosure are typically pharmaceutical compositions. Pharmaceutical compositions according to the present disclosure, and for use in accordance with the present disclosure, may comprise, in addition to the active ingredient(s), (i.e. AXL inhibitors and/or RAS inhibitor), a pharmaceutically acceptable excipient, carrier, buffer, stabiliser or other materials well known to those skilled in the art. Such materials should be non-toxic and should not interfere with the efficacy of the active ingredient(s). The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

In some aspects of the disclosure, the disclosed AXL inhibitor, RAS inhibitor, or AXL inhibitor+RAS inhibitor agent combination, may be comprised in a pharmaceutical composition, optionally further comprising a pharmaceutically acceptable excipient.

The present disclosure also provides such compositions for use in a method of treating a fibrotic disorder, and use of such compositions in the the manufacture of a medicament for treating a disorder in a subject, wherein the treatment comprises a method of treatment according to the present disclosure.

### Subjects

The terms “subject”, “patient” and “individual” are used interchangeably herein. The subject may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human. The subject may be any of its forms of development, for example, a foetus. In some aspects, the subject is a human.

### Subject Selection

Also provided by the present disclosure are methods of selecting a subject for treatment with one or more of an AXL inhibitor and a RAS inhibitor—such methods include:

A method of selecting a subject for treatment with an AXL inhibitor, wherein a subject is selected for treatment if the subject has been, will be, or is being treated with a RAS inhibitor. In other aspects, a subject is selected for treatment if the subject has been treated with a RAS inhibitor. In some aspects, a subject is selected for treatment if the subject is being treated with a RAS inhibitor. In some aspects, a subject is selected for treatment if the subject will be treated with a RAS inhibitor.

A method of selecting a subject for treatment with a RAS inhibitor, wherein a subject is selected for treatment if the subject has been, will be, or is being treated with an AXL inhibitor. In some aspects, a subject is selected for treatment if the subject has been treated with an AXL inhibitor. In some aspects, a subject is selected for treatment if the subject is being treated with an AXL inhibitor. In other aspects, a subject is selected for treatment if the subject will be treated with an AXL inhibitor.

A method of selecting a subject for treatment in a method of treatment as disclosed herein, comprising: identifying subjects having an increased activity or expression of AXL; and, selecting thus identified subjects for treatment.

A method of selecting a subject for treatment in a method of treatment as disclosed herein, comprising: identifying subjects having a fibrotic disease, and having increased activity or expression of AXL; and, selecting thus identified subjects for treatment.

In some aspects, increased activity or expression of AXL may be determined in a sample derived from a subject. In some aspects, increased activity or expression of AXL is determined relative to a control. The skilled person is readily able to determine suitable controls against which to assess increased activity or expression of AXL—for example, the control may be a level of activity or expression of AXL in healthy subjects, or in subjects known to respond to or benefit from treatment with the combination therapies disclosed herein.

Increased expression or expression of AXL can be determined by any suitable method known in the art—for example, by determining the copy number of the gene encoding AXL relative to a control sample (wherein an increase in the copy number indicates an increased level of expression), or by determining the level of AXL mRNA or protein relative to a control sample.

In some aspects, the disclosed methods of selecting a subject for treatment further comprise administering to the subject a therapeutically effective amount of an AXL inhibitor and/or a RAS inhibitor as appropriate. Such methods form part of the disclosed method of treating a fibrotic disorder.

### Dosage

It will be appreciated by one of skill in the art that appropriate dosages of the AXL inhibitors, RAS inhibitors and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In some cases, the dosage of AXL inhibitor may be determined by the expression of a first marker such as AXL observed in a sample obtained from the subject. Thus, the level or localisation of expression of the first marker in the sample may be indicative that a higher or lower dose of AXL inhibitor is required. For example, a high expression level of the first marker may indicate that a higher dose of AXL inhibitor would be suitable. In some cases, a high expression level of the first marker may indicate a more aggressive therapy.

In some cases, the dosage of the RAS inhibitor may be determined by the expression of a second marker observed in a sample obtained from the subject. Thus, the level or localisation of expression of the second marker in the sample may be indicative that a higher or lower dose of RAS inhibitor is required. For example, a high expression level of the second marker may indicate that a higher dose of RAS inhibitor would be suitable. In some cases, a high expression level of the second marker may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In some aspects, each active compound is administered to a human subject according to the following dosage regime: about 100 mg, 3 times daily. In other aspects, each active compound is administered to a human subject according to the following dosage regime: about 150 mg, 2 times daily. In other aspects, each active compound is administered to a human subject according to the following dosage regime: about 200 mg, 2 times daily. In yet other aspects, each active compound is administered to a human subject according to the following dosage regime: about 50 or about 75 mg, 3 or 4 times daily. In other aspects, each active compound is administered to a human subject according to the following dosage regime: about 100 or about 125 mg, 2 times daily.

### Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind a first target protein (Miller et al (2003) Jour. of Immunology 170:4854-4861). Antibodies may be murine, human, humanized, chimeric, or derived from other species such as rabbit, goat, sheep, horse or camel.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by Complementarity Determining Regions (CDRs) on multiple antibodies. Each antibody that specifically binds to a different epitope has a different structure. Thus, one antigen may have more than one corresponding antibody. An antibody may comprise a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. lgG, IgE, IgM, IgD, and IgA), class (e.g. IgG1, lgG2, lgG3, lgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule. The immunoglobulins can be derived from any species, including human, murine, or rabbit origin.

“Antibody fragments” comprise a portion of a full length antibody, generally the antigen binding or variable region thereof. Examples of antibody fragments include Fab, Fab', F(ab')2, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant

DNA methods (see, U.S. pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, lgG4, IgA, and IgA2. The heavy-chain constant domains that correspond to the different classes of antibodies are called α, δ, ϵ, γ, and μ, respectively. The subunit structures and three-dimensional configurations of different classes of immunoglobulins are well known.

### Aspects of the Disclosure

Certain aspects of the disclosure are as follows:

In some aspects the AXLi is an antagonistic anti-Axl antibody (such as Tilvestamab) and the RAS inhibitor is and ACEi (such as Enalapril).

In some aspects the fibrotic disorder is kidney, liver; or lung fibrosis. In some particularly aspects the fibrotic disorder is kidney fibrosis.

In some aspects: the AXLi and RASi are administered to the subject no more than 1 week apart, such as no more than 24 hours apart.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments and aspects described above, many equivalent modifications and variations will be apparent to those skilled in the art when given this disclosure. Accordingly, the exemplary emboidiments and aspects of the invention set forth above are considered to be illustrative and not limiting. Various changes to the described aspects may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by the use of the antecedent “about,” it will be understood that the particular value forms another aspect. The term “about” in relation to a numerical value is optional and means for example +/− 10%.

SEQUENCES

| SEQ | | |
|---|---|---|
| ID # | Description* | Sequence |
| 1 | BGB149 VH | EVQLVESGGGLVQPGGSLRLSCAASGYSFTDFYINWVRQAPGKGLEWVA |
| | | RIFPGGDNTYYNEKFKGRFTLSADTSKSTAYLQMNSLRAEDTAVYYCARR |
| | | GLYYAMDYWGQGTLVTVSS |
| 2 | BGB149 VL | DIQMTQSPSSLSASVGDRVTITCRSSQSLVHSNGIPYLHWYQQKPGKAPKL |
| | | LIYRVSNRFSGVPSRFSGSGSGTDFTLTISSLQPEDFATYYCSQGTHVPPTF |
| | | GQGTKVEIK |
| 3 | BGB149 HCDR1[Ch] | GYSFTDF |
| 4 | BGB149 HCDR2[Ch] | FPGGDN |
| 5 | BGB149 HCDR3[Ch] | RGLYYAMDY |
| 6 | BGB149 LCDR1[Ch] | RSSQSLVHSNGIPYLH |
| 7 | BGB149 LCDR2[Ch] | RVSNRFS |
| 8 | BGB149 LCDR3[Ch] | SQGTHVPPT |
| 9 | BGB149 HCDR1[A] | GYSFTDFYIN |
| 10 | BGB149 HCDR2[A] | RIFPGGDNTY |
| 11 | BGB149 HCDR3[A] | RGLYYAMDY |
| 12 | BGB149 LCDR1[A] | RSSQSLVHSNGIPYLH |
| 13 | BGB149 LCDR2[A] | RVSNRFS |
| 14 | BGB149 LCDR3[A] | SQGTHVPPT |
| 15 | BGB149 HCDR1[K] | DFYIN |
| 16 | BGB149 HCDR2[K] | RIFPGGDNTYYNEKFKG |
| 17 | BGB149 HCDR3[K] | RGLYYAMDY |
| 18 | BGB149 LCDR1[K] | RSSQSLVHSNGIPYLH |
| 19 | BGB149 LCDR2[K] | RVSNRFS |
| 20 | BGB149 LCDR3[K] | SQGTHVPPT |
| 21 | BGB149 HCDR1[Co] | TDFYIN |
| 22 | BGB149 HCDR2[Co] | WVARIFPGGDNTY |
| 23 | BGB149 HCDR3[Co] | ARRGLYYAMD |
| 24 | BGB149 LCDR1[Co] | VHSNGIPYLHWY |
| 25 | BGB149 LCDR2[Co] | LLIYRVSNRF |
| 26 | BGB149 LCDR3[Co] | SQGTHVPP |
| 27 | BGB149 HCDR1[I] | GYSFTDFY |
| 28 | BGB149 HCDR2[I] | IFPGGDNT |
| 29 | BGB149 HCDR3[I] | ARRGLYYAMDY |
| 30 | BGB149 LCDR1[I] | QSLVHSNGIPY |
| 31 | BGB149 LCDR2[I] | RV |
| 32 | BGB149 LCDR3[I] | SQGTHVPPT |
| 33 | 10G5alt VH | EVQLVQSGAGLVQPGGSVRLSCAASGYSFTDFYINWVRQAPGKGLEWIAR |
| | | IFPGGDNTYYNEKFKGRFTLSADTSSSTAYLQLNSLRAEDTAVYYCARRGL |
| | | YYAMDYWGQGTLVTVSS |
| 34 | 10G5alt VL | DIQMTQSPSSLSASVGDRVTITCRSSQSLVHSNGIPYLHWYQQKPGKAPKL |
| | | LIYRVSNRFSGVPSRFSGSRSGTDFTLTISSLQPEDFATYYCSQGTHVPPTF |
| | | GQGTKVEIK |
| 35 | 10G5alt HCDR1[Ch] | GYSFTDF |
| 36 | 10G5alt HCDR2[Ch] | FPGGDN |
| 37 | 10G5alt HCDR3[Ch] | RGLYYAMDY |
| 38 | 10G5alt LCDR1[Ch] | RSSQSLVHSNGIPYLH |
| 39 | 10G5alt LCDR2[Ch] | RVSNRFS |
| 40 | 10G5alt LCDR3[Ch] | SQGTHVPPT |
| 41 | 10G5alt HCDR1[A] | GYSFTDFYIN |
| 42 | 10G5alt HCDR2[A] | RIFPGGDNTY |
| 43 | 10G5alt HCDR3[A] | RGLYYAMDY |
| 44 | 10G5alt LCDR1[A] | RSSQSLVHSNGIPYLH |
| 45 | 10G5alt LCDR2[A] | RVSNRFS |
| 46 | 10G5alt LCDR3[A] | SQGTHVPPT |
| 47 | 10G5alt HCDR1[K] | DFYIN |
| 48 | 10G5alt HCDR2[K] | RIFPGGDNTYYNEKFKG |
| 49 | 10G5alt HCDR3[K] | RGLYYAMDY |
| 50 | 10G5alt LCDR1[K] | RSSQSLVHSNGIPYLH |
| 51 | 10G5alt LCDR2[K] | RVSNRFS |
| 52 | 10G5alt LCDR3[K] | SQGTHVPPT |
| 53 | 10G5alt HCDR1[Co] | TDFYIN |
| 54 | 10G5alt HCDR2[Co] | WIARIFPGGDNTY |
| 55 | 10G5alt HCDR3[Co] | ARRGLYYAMD |
| 56 | 10G5alt LCDR1[Co] | VHSNGIPYLHWY |
| 57 | 10G5alt LCDR2[Co] | LLIYRVSNRF |
| 58 | 10G5alt LCDR3[Co] | SQGTHVPP |
| 59 | 10G5alt HCDR1[I] | GYSFTDFY |
| 60 | 10G5alt HCDR2[I] | IFPGGDNT |
| 61 | 10G5alt HCDR3[I] | ARRGLYYAMDY |
| 62 | 10G5alt LCDR1[I] | QSLVHSNGIPY |
| 63 | 10G5alt LCDR2[I] | RV |
| 64 | 10G5alt LCDR3[I] | SQGTHVPPT |
| 65 | m10C9 VH | QVQLQQPGPELVKPGASVKISCKTSDYNFTRYYIHWVKQRPGQGLEWIGW |
| | | IYPGTGDSKYNEKFKGRATLTADTSSSTAYMQLSSQTSEDSAVYFCARNG |
| | | NYWYFDVWGAGTAVTVSS |
| 66 | m10C9 VL | DIVMTQAAPSGPVTPGESVSISCRSSKSLLHSNGNTYLYWFLQRPGQSPQL |
| | | LIYRMSNLASGVPDRFSGSGSGTAFTLRISRVEAEDVGIYYCMQHREYPFT |
| | | FGGGTKLEIK |
| 67 | m10C9 HCDR1[Ch] | DYNFTRY |
| 68 | m10C9 HCDR2[Ch] | YPGTGD |
| 69 | m10C9 HCDR3[Ch] | NGNYWYFDV |
| 70 | m10C9 LCDR1[Ch] | RSSKSLLHSNGNTYLY |
| 71 | m10C9 LCDR2[Ch] | RMSNLAS |
| 72 | m10C9 LCDR3[Ch] | MQHREYPFT |
| 73 | m10C9 HCDR1[A] | DYNFTRYYIH |
| 74 | m10C9 HCDR2[A] | WIYPGTGDSK |
| 75 | m10C9 HCDR3[A] | NGNYWYFDV |
| 76 | m10C9 LCDR1[A] | RSSKSLLHSNGNTYLY |
| 77 | m10C9 LCDR2[A] | RMSNLAS |
| 78 | m10C9 LCDR3[A] | MQHREYPFT |
| 79 | m10C9 HCDR1[K] | RYYIH |
| 80 | m10C9 HCDR2[K] | WIYPGTGDSKYNEKFKG |
| 81 | m10C9 HCDR3[K] | NGNYWYFDV |
| 82 | m10C9 LCDR1[K] | RSSKSLLHSNGNTYLY |
| 83 | m10C9 LCDR2[K] | RMSNLAS |
| 84 | m10C9 LCDR3[K] | MQHREYPFT |
| 85 | m10C9 HCDR1[Co] | TRYYIH |
| 86 | m10C9 HCDR2[Co] | WIGWIYPGTGDSK |
| 87 | m10C9 HCDR3[Co] | ARNGNYWYFD |
| 88 | m10C9 LCDR1[Co] | LHSNGNTYLYWF |
| 89 | m10C9 LCDR2[Co] | LLIYRMSNLA |
| 90 | m10C9 LCDR3[Co] | MQHREYPF |
| 91 | m10C9 HCDR1[I] | DYNFTRYY |
| 92 | m10C9 HCDR2[I] | IYPGTGDS |
| 93 | m10C9 HCDR3[I] | ARNGNYWYFDV |
| 94 | m10C9 LCDR1[I] | KSLLHSNGNTY |
| 95 | m10C9 LCDR2[I] | RM |
| 96 | m10C9 LCDR3[I] | MQHREYPFT |
| 97 | YW327.6S2 VH | EVQLVESGGGLVQPGGSLRLSCAASGFSLSGSWIHWVRQAPGKGLEWVG |
| | | WINPYRGYAYYADSVKGRFTISADTSKNTAYLQMNSLRAEDTAVYYCAREY |
| | | SGWGGSSVGYAMDYWGQGTLVTVSS |
| 98 | YW327.6S2 VL | DIQMTQSPSSLSASVGDRVTITCRASQDVSTAVAWYQQKPGKAPKLLIYSA |
| | | SFLYSGVPSRFSGSGSGTDFTLTISSLQPEDFATYYCQQSYTTPPTFGQGT |
| | | KVEIKR |
| 99 | YW327.6S2 HCDR1[Ch] | GFSLSGS |
| 100 | YW327.6S2 HCDR2[Ch] | NPYRGY |
| 101 | YW327.6S2 | EYSGWGGSSVGYAMDY |
| | HCDR3[Ch] | |
| 102 | YW327.6S2 LCDR1[Ch] | RASQDVSTAVA |
| 103 | YW327.6S2 LCDR2[Ch] | SASFLYS |
| 104 | YW327.6S2 LCDR3[Ch] | QQSYTTPPT |
| 105 | YW327.6S2 HCDR1[A] | GFSLSGSWIH |
| 106 | YW327.6S2 HCDR2[A] | WINPYRGYAY |
| 107 | YW327.6S2 HCDR3[A] | EYSGWGGSSVGYAMDY |
| 108 | YW327.6S2 LCDR1[A] | RASQDVSTAVA |
| 109 | YW327.6S2 LCDR2[A] | SASFLYS |
| 110 | YW327.6S2 LCDR3[A] | QQSYTTPPT |
| 111 | YW327.6S2 HCDR1[K] | GSWIH |
| 112 | YW327.6S2 HCDR2[K] | WINPYRGYAYYADSVKG |
| 113 | YW327.6S2 HCDR3[K] | EYSGWGGSSVGYAMDY |
| 114 | YW327.6S2 LCDR1[K] | RASQDVSTAVA |
| 115 | YW327.6S2 LCDR2[K] | SASFLYS |
| 116 | YW327.6S2 LCDR3[K] | QQSYTTPPT |
| 117 | YW327.6S2 HCDR1[Co] | SGSWIH |
| 118 | YW327.6S2 HCDR2[Co] | WVGWINPYRGYAY |
| 119 | YW327.6S2 HCDR3[Co] | AREYSGWGGSSVGYAMD |
| 120 | YW327.6S2 LCDR1[Co] | STAVAWY |
| 121 | YW327.6S2 LCDR2[Co] | LLIYSASFLY |
| 122 | YW327.6S2 LCDR3[Co] | QQSYTTPP |
| 123 | YW327.6S2 HCDR1[I] | GFSLSGSW |
| 124 | YW327.6S2 HCDR2[I] | INPYRGYA |
| 125 | YW327.6S2 HCDR3[I] | AREYSGWGGSSVGYAMDY |
| 126 | YW327.6S2 LCDR1[I] | QDVSTA |
| 127 | YW327.6S2 LCDR2[I] | SA |
| 128 | YW327.6S2 LCDR3[I] | QQSYTTPPT |
| 129 | 1003A2 VH | EVKLVESGGGLVQPGGSLKLSCAASGFTFNSYSMSWVRQTPEKRLEWVA |
| | | YISNGGGRTYYPDTIKGRFTLSRDNDRNTLYLQMSSLKSEDTAMYYCARGT |
| | | TESYYALDYWGQGTSVTVSS |
| 130 | 1003A2 VL | DIVMTQSHKFMSTSLGDRISITCKASQDVITAVAWYQQKPGQSPKLLIYWAS |
| | | TRHTGVPDRFTGSGSGTDYNLTISSVQAEDLALYYCQQHFSTPLTFGAGTK |
| | | LELK |
| 131 | 1003A2 HCDR1[Ch] | GFTFNSY |
| 132 | 1003A2 HCDR2[Ch] | SNGGGR |
| 133 | 1003A2 HCDR3[Ch] | GTTESYYALDY |
| 134 | 1003A2 LCDR1[Ch] | KASQDVITAVA |
| 135 | 1003A2 LCDR2[Ch] | WASTRHT |
| 136 | 1003A2 LCDR3[Ch] | QHFSTPLT |
| 137 | 1003A2 HCDR1[A] | GFTFNSYSMS |
| 138 | 1003A2 HCDR2[A] | YISNGGGRTY |
| 139 | 1003A2 HCDR3[A] | GTTESYYALDY |
| 140 | 1003A2 LCDR1[A] | KASQDVITAVA |
| 141 | 1003A2 LCDR2[A] | WASTRHT |
| 142 | 1003A2 LCDR3[A] | QQHFSTPLT |
| 143 | 1003A2 HCDR1[K] | SYSMS |
| 144 | 1003A2 HCDR2[K] | YISNGGGRTYYPDTIKG |
| 145 | 1003A2 HCDR3[K] | GTTESYYALDY |
| 146 | 1003A2 LCDR1[K] | KASQDVITAVA |
| 147 | 1003A2 LCDR2[K] | WASTRHT |
| 148 | 1003A2 LCDR3[K] | QQHFSTPLT |
| 149 | 1003A2 HCDR1[Co] | NSYSMS |
| 150 | 1003A2 HCDR2[Co] | WVAYISNGGGRTY |
| 151 | 1003A2 HCDR3[Co] | ARGTTESYYALD |
| 152 | 1003A2 LCDR1[Co] | ITAVAWY |
| 153 | 1003A2 LCDR2[Co] | LLIYWASTRH |
| 154 | 1003A2 LCDR3[Co] | QQHFSTPL |
| 155 | 1003A2 HCDR1[I] | GFTFNSYS |
| 156 | 1003A2 HCDR2[I] | ISNGGGRT |
| 157 | 1003A2 HCDR3[I] | ARGTTESYYALDY |
| 158 | 1003A2 LCDR1[I] | QDVITA |
| 159 | 1003A2 LCDR2[I] | WA |
| 160 | 1003A2 LCDR3[I] | QQHFSTPLT |
| 161 | 1613F12 VH | EVHLQQSGAELVKPGASVKLSCTASGFNIRDTYIHWVKQRPEQGLEWIGRL |
| | | DPANGHTKYGPNFQGRATMTSDTSSNTAYLQLSSLTSEDTAVYYCARGAY |
| | | YYGSSGLFYFDYWGQGTTLSVSS |
| 162 | 1613F12 VL | DVQITQSPSYLATSPGETITINCRASKSISKYLAWYQEKPGKTNKLLIYSGST |
| | | LQSGVPSRFSGSGSGTDFTLTISSLEPEDFAMYFCQQHHEYPLTFGAGTEL |
| | | ELK |
| 163 | 1613F12 HCDR1[Ch] | GFNIRDT |
| 164 | 1613F12 HCDR2[Ch] | DPANGH |
| 165 | 1613F12 HCDR3[Ch] | GAYYYGSSGLFYFDY |
| 166 | 1613F12 LCDR1[Ch] | RASKSISKYLA |
| 167 | 1613F12 LCDR2[Ch] | SGSTLQS |
| 168 | 1613F12 LCDR3[Ch] | QQHHEYPLT |
| 169 | 1613F12 HCDR1[A] | GFNIRDTYIH |
| 170 | 1613F12 HCDR2[A] | RLDPANGHTK |
| 171 | 1613F12 HCDR3[A] | GAYYYGSSGLFYFDY |
| 172 | 1613F12 LCDR1[A] | RASKSISKYLA |
| 173 | 1613F12 LCDR2[A] | SGSTLQS |
| 174 | 1613F12 LCDR3[A] | QQHHEYPLT |
| 175 | 1613F12 HCDR1[K] | DTYIH |
| 176 | 1613F12 HCDR2[K] | RLDPANGHTKYGPNFQG |
| 177 | 1613F12 HCDR3[K] | GAYYYGSSGLFYFDY |
| 178 | 1613F12 LCDR1[K] | RASKSISKYLA |
| 179 | 1613F12 LCDR2[K] | SGSTLQS |
| 180 | 1613F12 LCDR3[K] | QQHHEYPLT |
| 181 | 1613F12 HCDR1[Co] | RDTYIH |
| 182 | 1613F12 HCDR2[Co] | WIGRLDPANGHTK |
| 183 | 1613F12 HCDR3[Co] | ARGAYYYGSSGLFYFD |
| 184 | 1613F12 LCDR1[Co] | SKYLAWY |
| 185 | 1613F12 LCDR2[Co] | LLIYSGSTLQ |
| 186 | 1613F12 LCDR3[Co] | QQHHEYPL |
| 187 | 1613F12 HCDR1[I] | GFNIRDTY |
| 188 | 1613F12 HCDR2[I] | LDPANGHT |
| 189 | 1613F12 HCDR3[I] | ARGAYYYGSSGLFYFDY |
| 190 | 1613F12 LCDR1[I] | KSISKY |
| 191 | 1613F12 LCDR2[I] | SG |
| 192 | 1613F12 LCDR3[I] | QQHHEYPLT |
| 193 | 110D7 VH | EVKLVESGGGLVQPGGSLKLSCATSGFTFSDYYMYWVRQTPEKRLEWVA |
| | | YISNGGGSTYYPDTVKGRFTMSRDNAKNTLYLQMSRLKSEDTAMYYCARL |
| | | SFYAMDYWGQGTSVTVSS |
| 194 | 110D7 VL | DIVMTQSHKFMSTSVGDRVSITCKASQDVTTAVAWYQQKPGQSPKLLIYW |
| | | ASTRHTGVPDRFTGSGSGTDYTLTISSVQAEDLALYYCQQHYRIPFTFGSG |
| | | TKLEIK |
| 195 | 110D7 HCDR1[Ch] | GFTFSDY |
| 196 | 110D7 HCDR2[Ch] | SNGGGS |
| 197 | 110D7 HCDR3[Ch] | LSFYAMDY |
| 198 | 110D7 LCDR1[Ch] | KASQDVTTAVA |
| 199 | 110D7 LCDR2[Ch] | WASTRHT |
| 200 | 110D7 LCDR3[Ch] | QQHYRIPFT |
| 201 | 110D7 HCDR1[A] | GFTFSDYYMY |
| 202 | 110D7 HCDR2[A] | YISNGGGSTY |
| 203 | 110D7 HCDR3[A] | LSFYAMDY |
| 204 | 110D7 LCDR1[A] | KASQDVTTAVA |
| 205 | 110D7 LCDR2[A] | WASTRHT |
| 206 | 110D7 LCDR3[A] | QQHYRIPFT |
| 207 | 110D7 HCDR1[K] | DYYMY |
| 208 | 110D7 HCDR2[K] | YISNGGGSTYYPDTVKG |
| 209 | 110D7 HCDR3[K] | LSFYAMDY |
| 210 | 110D7 LCDR1[K] | KASQDVTTAVA |
| 211 | 110D7 LCDR2[K] | WASTRHT |
| 212 | 110D7 LCDR3[K] | QQHYRIPFT |
| 213 | 110D7 HCDR1[Co] | SDYYMY |
| 214 | 110D7 HCDR2[Co] | WVAYISNGGGSTY |
| 215 | 110D7 HCDR3[Co] | ARLSFYAMD |
| 216 | 110D7 LCDR1[Co] | TTAVAWY |
| 217 | 110D7 LCDR2[Co] | LLIYWASTRH |
| 218 | 110D7 LCDR3[Co] | QQHYRIPF |
| 219 | 110D7 HCDR1[I] | GFTFSDYY |
| 220 | 110D7 HCDR2[I] | ISNGGGST |
| 221 | 110D7 HCDR3[I] | ARLSFYAMDY |
| 222 | 110D7 LCDR1[I] | QDVTTA |
| 223 | 110D7 LCDR2[I] | WA |
| 224 | 110D7 LCDR3[I] | QQHYRIPFT |
| 225 | 1024G11 VH | EVKLVESGGGLVQPGGSLNLSCAASGFTFSSYSMSWVRQTPEKRLEWVA |
| | | FISNGGGRTYYPDTVKGRFTFSRDNARNTLYLQMSSLNSEDTAMYFCARG |
| | | TTESYYAMDYWGQGTSVTVSS |
| 226 | 1024G11 VL | DIVMTQSHKFMSTSVGDRVSITCKASQDVITAVAWYQQKPGQSPKLLIYWA |
| | | STRHTGVPDRFTGSGSGTDYTLTISSVQAEDLALYFCQQHLSTPLTFGAGT |
| | | KLELK |
| 227 | 1024G11 HCDR1[Ch] | GFTFSSY |
| 228 | 1024G11 HCDR2[Ch] | SNGGGR |
| 229 | 1024G11 HCDR3[Ch] | GTTESYYAMDY |
| 230 | 1024G11 LCDR1[Ch] | KASQDVITAVA |
| 231 | 1024G11 LCDR2[Ch] | WASTRHT |
| 232 | 1024G11 LCDR3[Ch] | QQHLSTPLT |
| 233 | 1024G11 HCDR1[A] | GFTFSSYSMS |
| 234 | 1024G11 HCDR2[A] | FISNGGGRTY |
| 235 | 1024G11 HCDR3[A] | GTTESYYAMDY |
| 236 | 1024G11 LCDR1[A] | KASQDVITAVA |
| 237 | 1024G11 LCDR2[A] | WASTRHT |
| 238 | 1024G11 LCDR3[A] | QQHLSTPLT |
| 239 | 1024G11 HCDR1[K] | SYSMS |
| 240 | 1024G11 HCDR2[K] | FISNGGGRTYYPDTVKG |
| 241 | 1024G11 HCDR3[K] | GTTESYYAMDY |
| 242 | 1024G11 LCDR1[K] | KASQDVITAVA |
| 243 | 1024G11 LCDR2[K] | WASTRHT |
| 244 | 1024G11 LCDR3[K] | QQHLSTPLT |
| 245 | 1024G11 HCDR1[Co] | SSYSMS |
| 246 | 1024G11 HCDR2[Co] | WVAFISNGGGRTY |
| 247 | 1024G11 HCDR3[Co] | ARGTTESYYAMD |
| 248 | 1024G11 LCDR1[Co] | ITAVAWY |
| 249 | 1024G11 LCDR2[Co] | LLIYWASTRH |
| 250 | 1024G11 LCDR3[Co] | QQHLSTPL |
| 251 | 1024G11 HCDR1[I] | GFTFSSYS |
| 252 | 1024G11 HCDR2[I] | ISNGGGRT |
| 253 | 1024G11 HCDR3[I] | ARGTTESYYAMDY |
| 254 | 1024G11 LCDR1[I] | QDVITA |
| 255 | 1024G11 LCDR2[I] | WA |
| 256 | 1024G11 LCDR3[I] | QQHLSTPLT |

*For CDR sequences, [Ch] = Chothia definition, [A] = AbM definition, [K] = Kabat definition, [Co] = Contact definition, [I] = IMGT definition

### STATEMENTS OF DISCLOSURE

The following numbered statements, outlining aspects of the present disclosure, are part of the description.

101. A method of treating a fibrotic disease, the method comprising administering to a subject in need thereof a therapeutically effective amount of an AXL inhibitor (AXLi), wherein the AXL inhibitor is administered in combination with a RAS inhibitor.

### AXL Inhibitor

102. The method of statement 101, wherein the AXL inhibitor is an antibody that binds AXL.

103. The method of statement 102, wherein the AXL inhibitor is an antibody that binds human AXL.

104. The method of either one of statements 102 or 103, wherein the anti-AXL antibody is an antibody as described in WO/2016/097370, WO/2017/220695, WO/2015/193428, WO/2016/166296, WO/2015/193430, EP2267454, WO/2009/063965, WO/2011/159980, WO/2012/175691, WO/2012/175692, WO/2013/064685, WO/2014/068139, WO/2009/062690, WO/2020/205576, or WO/2010/130751.

105. The method of either one of statements 102 or 103, wherein the anti-AXL antibody antagonises Axl activity.

106. The method of statement 105, wherein the anti-AXL antibody inhibits Axl kinase and/or signalling activity.

107. The method of either one of statements 105 or 106, wherein the anti-AXL antibody inhibits the binding of AXL to the GAS6 ligand.

108. The method of any one of statements 102 to 107, wherein the anti-AXL antibody crosslinks Axl receptors.

109. The method of any one of statements 102 to 108, wherein the anti-AXL antibody comprises the CDRs of the VH domain having the sequence of SEQ ID No. 1 and/or the CDRs of VL domain having the sequence set out herein as SEQ ID No.2.

110. The method of statement 109, wherein the anti-AXL antibody comprises the 6 CDRs having the sequences set out herein as: SEQ ID Nos. 3 to 8, SEQ ID Nos. 9 to 14, SEQ ID Nos. 15 to 20, SEQ ID Nos. 21 to 26, or SEQ ID Nos. 27 to 32.

111. The method of statement 109, wherein the anti-AXL antibody comprises a VH domain having the sequence of SEQ ID No. 1 and/or a VL domain having the sequence of SEQ ID No.2.

112. The method of any one of statements 102 to 108, wherein the anti-AXL antibody is Tilvestamab.

113. The method of any one of statements 102 to 108, wherein the anti-AXL antibody comprises the CDRs of the VH domain having the sequence of SEQ ID No.33 and/or the CDRs of VL domain having the sequence set out herein as SEQ ID No.34.

114. The method of statement 113, wherein the anti-AXL antibody comprises the 6 CDRs having the sequences set out herein as: SEQ ID Nos. 35 to 40, SEQ ID Nos. 41 to 46, SEQ ID Nos. 47 to 52, SEQ ID Nos. 53 to 58, or SEQ ID Nos. 59 to 64.

115. The method of statement 113, wherein the anti-AXL antibody comprises a VH domain having the sequence of SEQ ID No.33 and/or a VL domain having the sequence of SEQ ID No.34.

116. The method of any one of statements 102 to 108, wherein the anti-AXL antibody comprises the CDRs of the VH domain having the sequence of SEQ ID No.65 and/or the CDRs of VL domain having the sequence set out herein as SEQ ID No.66.

117. The method of statement 116, wherein the anti-AXL antibody comprises the 6 CDRs having the sequences set out herein as: SEQ ID Nos. 67 to 72, SEQ ID Nos. 73 to 78, SEQ ID Nos. 79 to 84, SEQ ID Nos. 85 to 90, or SEQ ID Nos. 91 to 96.

118. The method of statement 116, wherein the anti-AXL antibody comprises a VH domain having the sequence of SEQ ID No.65 and/or a VL domain having the sequence of SEQ ID No.66.

119. The method of any one of statements 102 to 108, wherein the anti-AXL antibody comprises the CDRs of the VH domain having the sequence of SEQ ID No.97 and/or the CDRs of VL domain having the sequence set out herein as SEQ ID No.98.

120. The method of statement 119, wherein the anti-AXL antibody comprises the 6 CDRs having the sequences set out herein as: SEQ ID Nos. 99 to 104, SEQ ID Nos. 105 to 110, SEQ ID Nos. 111 to 116, SEQ ID Nos. 117 to 122, or SEQ ID Nos. 123 to 128.

121. The method of statement 119, wherein the anti-AXL antibody comprises a VH domain having the sequence of SEQ ID No.97 and/or a VL domain having the sequence of SEQ ID No.98.

122. The method of any one of statements 102 to 108, wherein the anti-AXL antibody comprises the CDRs of the VH domain having the sequence of SEQ ID No. 129 and/or the CDRs of VL domain having the sequence set out herein as SEQ ID No. 130.

123. The method of statement 122, wherein the anti-AXL antibody comprises the 6 CDRs having the sequences set out herein as: SEQ ID Nos. 131 to 136, SEQ ID Nos. 137 to 142, SEQ ID Nos. 143 to 148, SEQ ID Nos. 149 to 154, or SEQ ID Nos. 155 to 160.

124. The method of statement 122, wherein the anti-AXL antibody comprises a VH domain having the sequence of SEQ ID No. 129 and/or a VL domain having the sequence of SEQ ID No.130.

125. The method of any one of statements 102 to 108, wherein the anti-AXL antibody comprises the CDRs of the VH domain having the sequence of SEQ ID No. 161 and/or the CDRs of VL domain having the sequence set out herein as SEQ ID No. 162.

126. The method of statement 125, wherein the anti-AXL antibody comprises the 6 CDRs having the sequences set out herein as: SEQ ID Nos. 163 to 168, SEQ ID Nos. 169 to 174, SEQ ID Nos. 175 to 180, SEQ ID Nos. 181 to 186, or SEQ ID Nos. 187 to 192.

127. The method of statement 125, wherein the anti-AXL antibody comprises a VH domain having the sequence of SEQ ID No.161 and/or a VL domain having the sequence of SEQ ID No.162.

128. The method of any one of statements 102 to 108, wherein the anti-AXL antibody comprises the CDRs of the VH domain having the sequence of SEQ ID No. 193 and/or the CDRs of VL domain having the sequence set out herein as SEQ ID No. 194.

129. The method of statement 128, wherein the anti-AXL antibody comprises the 6 CDRs having the sequences set out herein as: SEQ ID Nos. 195 to 200, SEQ ID Nos. 201 to 206, SEQ ID Nos. 207 to 212, SEQ ID Nos. 213 to 218, or SEQ ID Nos. 219 to 224.

130. The method of statement 128, wherein the anti-AXL antibody comprises a VH domain having the sequence of SEQ ID No. 193 and/or a VL domain having the sequence of SEQ ID No.194.

131. The method of any one of statements 102 to 108, wherein the anti-AXL antibody comprises the CDRs of the VH domain having the sequence of SEQ ID No.225 and/or the CDRs of VL domain having the sequence set out herein as SEQ ID No.226.

132. The method of statement 131, wherein the anti-AXL antibody comprises the 6 CDRs having the sequences set out herein as: SEQ ID Nos. 227 to 232, SEQ ID Nos. 233 to 238, SEQ ID Nos. 239 to 244, SEQ ID Nos. 245 to 250, or SEQ ID Nos. 251 to 256.

133. The method of statement 131, wherein the anti-AXL antibody comprises a VH domain having the sequence of SEQ ID No.225 and/or a VL domain having the sequence of SEQ ID No.226.

134. The method of statement 101, wherein the AXL inhibitor is bemcentinib (BGB324 / R428).

135. The method of statement 101, wherein the AXL inhibitor is selected from the group consisting of: dubermatinib (CAS No.1341200-45-0; UNII 14D65TV20J); gilteritinib (CAS No. 1254053-43-4; UNII 66D92MGC8M); cabozantinib (CAS No. 849217-68-1; UNII 1C39JW444G); SGI7079 (CAS No. 1239875-86-5); merestinib (CAS No. 1206799-15-6; UNII 50GS5K699E); amuvatinib (CAS No. 850879-09-3; UNII SO9S6QZB4R); bosutinib (CAS No. 380843-75-4; UNII 5018V4AEZ0); glesatinib (CAS No. 936694-12-1; UNII 7Q290XD98N); foretinib (CAS No. 849217-64-7; UNII 81FH7VK1C4); and, TP0903 (CAS No. 1341200-45-0). cl RAS inhibitors

136. The method of any one of statements 101 to 135, wherein the RAS inhibitor is an ACE inhibitor.

137. The method of statement 136, wherein the ACE inhibitor is selected from the group consisting of Enalapril, Alacepril, Benazepril, Captopril, Cilazapril, Fosinopril, Imidapril, Lisinopril, Moexipril, Perindopril, Quinapril, Ramipril, Trandolapril, and Zofenopril.

138. The method of statement 136, wherein the ACE inhibitor is Enalapril.

139. The method of any one of statements 101 to 135, wherein the RAS inhibitor is an ARB.

140. The method of statement 139, wherein the ARB inhibitor is selected from the group consisting of Azilsartan, Candesartan, Eprosartan, Fimasartan, Irbesartan, Losartan, Olmesartan, Saprisartan, Telmisartan, and Valsartan.

141. The method of any one of statements 101 to 135, wherein the RAS inhibitor is an AA.

142. The method of statement 141, wherein the AA inhibitor is selected from the group consisting of Spironolactone, Eplerenone, Canrenone, Finerenone, and Mexrenone.

### Fibrotic Disorder

143. The method of any one of statements 101 to 142, wherein the fibrotic disorder is selected from the group consisting of: arthrofibrosis, Crohn's disease, Dupuytren's contracture, peyronie's disease, strabmisus, scleroderma, keloid, Nephrogenic systemic fibrosis, pulmonary fibrosis, idiopathic pulmonary fibrosis (IPF), cystic fibrosis (CF), progressive massive fibrosis, fibrothorax, systemic sclerosis, cardiac fibrosis, non-alcoholic steatohepatitis (NASH), other types of liver fibrosis, primary biliary cirrhosis, renal fibrosis, reteroperitoneal fibrosis, mediastinal fibrosis, myelofibrosis, and atherosclerosis.

144. The method of any one of statements 101 to 142, wherein the fibrotic disorder is selected from the group consisting of lung fibrosis, cardiac fibrosis, liver fibrosis, and kidney fibrosis.

145. The method of any one of statements 101 to 142, wherein the fibrotic disorder is kidney fibrosis.

### Administration Schedule Features

146. The method of any one of statements 101 to 145, wherein the AXL inhibitor is administered concurrently with the RAS inhibitor.

147. The method of any one of statements 101 to 145, wherein the AXL inhibitor is administered separately and/or sequentially to the RAS inhibitor.

148. The method of any one of statements 101 to 145, wherein the RAS inhibitor is administered subsequent to administration of the AXL inhibitor.

149. The method of any one of statements 101 to 145, wherein the AXL inhibitor is administered subsequent to administration of the RAS inhibitor.

150. The method of any one of statements 101 to 149, wherein the method comprises administering the AXL inhibitor to the subject, wherein the RAS inhibitor has been, is, or will be, administered to the subject.

151. The method of any one of statements 101 to 150, wherein the method comprises administering the AXL inhibitor and/or RAS inhibitor alone or in further combination with other treatments.

201. An AXL inhibitor and a RAS inhibitor for use in a method of treating a Fibrotic disorder according to any one of statements 101 to 151.

202. An AXL inhibitor for use in a method of treating a fibrotic disorder according to any one of statements 101 to 151, the method comprising administering to a subject in need thereof a therapeutically effective amount of an AXL inhibitor, wherein the AXL inhibitor is administered in combination with a RAS inhibitor.

203. A RAS inhibitor for use in a method of treating a fibrotic disorder according to any one of statements 101 to 151, the method comprising administering to a subject in need thereof a therapeutically effective amount of a RASi, wherein the RASi is administered in combination with an AXL inhibitor.

301. Use of an AXL inhibitor and a RAS inhibitor in the manufacture of a medicament for treating a disorder in a subject, wherein the treatment comprises a method of treating a fibrotic disorder according to any one of statements 101 to 151.

302. Use of an AXL inhibitor in the manufacture of a medicament for treating a disorder in a subject, wherein the treatment comprises a method of treating a fibrotic disorder according to any one of statements 101 to 151.

303. Use of a RAS inhibitor in the manufacture of a medicament for treating a disorder in a subject, wherein the treatment comprises a method of treating a fibrotic disorder according to any one of statements 101 to 151.

401. A kit comprising an AXL inhibitor and a RAS inhibitor for use in a method of treating a fibrotic disorder according to any one of statements 101 to 151.

501. A pharmaceutical composition comprising: an AXL inhibitor; a RAS inhibitor; and, a pharmaceutically acceptable excipient.

502. A method of treating a fibrotic disorder, the method comprising administering to a subject in need thereof a therapeutically effective amount of an AXL inhibitor in concurrent, separate, or sequential combination with a RAS inhibitor.

503. A method of treating a fibrotic disorder, the method comprising administering to a subject in need thereof a therapeutically effective amount of an AXL inhibitor,
wherein the subject has been, will be, or is being treated with a RAS inhibitor.

504. A method of treating a fibrotic disorder, the method comprising administering to a subject in need thereof a therapeutically effective amount of a RAS inhibitor,
wherein the subject has been, will be, or is being treated with an AXL inhibitor.

505. A method of selecting a subject for treatment with an AXL inhibitor, wherein a subject is selected for treatment if the subject has been, will be, or is being treated with a RAS inhibitor.

506. A method of selecting a subject for treatment with a RAS inhibitor,
wherein a subject is selected for treatment if the subject has been, will be, or is being treated with an AXL inhibitor.

507. The method of any one of statements 501 to 506, further comprising administering to the subject a therapeutically effective amount of an AXL inhibitor and/or a RAS inhibitor.

### EXAMPLES

### Example 1

### Background

Precision Cut Kidney Slices (PCKS) from human kidney tissue are physiologically and structurally representative of the tissue architecture. Testing potential therapeutic targets and interrogating mechanisms underpinning disease pathophysiology in human PCKS allows the assessment of the targets effectiveness and relevance to the clinical situation, overcoming many of the limitations of currently widely employed in vivo rodent models and in vitro 2D cell culture methodologies.

To date the main problem with using human PCKS has been the loss of metabolic and structural viability of the tissue within a relatively short timeframe, reported to be as little as 8 hrs in some studies. The assyas described below utilise a novel tissue bioreactor technology that maintains viability and functionality of PCKS from human kidney tissue for at least 6 days in vitro allowing sufficient time to model fibrosis ex vivo.

### Materials & Methods

PCKS preparation and culture

PCKS were prepared from explanted kidney tissue and rested for 24 hours to allow the post-slicing stress period to elapse before experiments began. PCKS were cultured without exogenous challenge in the presence or absence of novel inhibitors as indicated in the table below. All PCKS were harvested at 96 hrs.

### Study Groups

Groups with n=6 human PCKS (total n=108 per donor) were investigated as shown below. PCKS were prepared from n=1 individual human kidney. The human kidneys are unused donor kidneys that are assessed but not utilised for transplant.

TABLE 1
experimental groups

| Group | Intervention | Treatment dose | | Rest | Harvest | PCKS |
|---|---|---|---|---|---|---|
| 1 | Vehicle | DMSO | | 24 hrs | 96 hrs | n = 6 |
| 2 | ALK5i | 10 | μM | 24 hrs | 96 hrs | n = 6 |
| 3 | Bemcentinib (DMSO) | 0.11 | μM | 24 hrs | 96 hrs | n = 6 |
| 4 | Bemcentinib (DMSO) | 0.33 | μM | 24 hrs | 96 hrs | n = 6 |
| 5 | Bemcentinib (DMSO) | 1 | μM | 24 hrs | 96 hrs | n = 6 |
| 6 | Bemcentinib (DMSO) | 3 | μM | 24 hrs | 96 hrs | n = 6 |
| 7 | Nintedanib (DMSO) | 4 | μM | 24 hrs | 96 hrs | n = 6 |
| 8 | ASK1 inhibitor Selonsertib (DMSO) | 500 ng/ml (1.1 uM) | | 24 hrs | 96 hrs | n = 6 |
| 9 | ACE inhibitor Enalapril (DMSO) | 0.75 | μM | 24 hrs | 96 hrs | n = 6 |
| 10 | ACE inhibitor Enalapril (DMSO) | 0.15 | μM | 24 hrs | 96 hrs | n = 6 |
| 11 | Bemcentinib + ACE inhibitor | 0.33 μM + | | 24 hrs | 96 hrs | n = 6 |
| | Enalapril (DMSO) | 0.15 μM | | | | |
| 12 | control IgG1 | 125 | μg/mL | 24 hrs | 96 hrs | n = 6 |
| 13 | tilvestamab | 1 | μg/mL | 24 hrs | 96 hrs | n = 6 |
| 14 | tilvestamab | 5 | μg/mL | 24 hrs | 96 hrs | n = 6 |
| 15 | tilvestamab | 2 | μg/mL | 24 hrs | 96 hrs | n = 6 |
| 16 | tilvestamab | 125 | μg/mL | 24 hrs | 96 hrs | n = 6 |

| | | | | | |
|---|---|---|---|---|---|
| 17 | control IgG1 + DMSO | 1 μg/mL + DMSO | 24 hrs | 96 hrs | n = 6 |
| 18 | Tilvestamab + ACE inhibitor | 1 μg/mL + | 24 hrs | 96 hrs | n = 6 |
| | Enalapril (DMSO) | 0.15 μM | | | |

### PCKS Treatments and Media Collections

PCKS were incubated for a 24 hr rest period. Post-rest, PCKS were incubated for a further 72 hrs in the presence or absence of inhibitors. PCKS culture media, including all compounds, was refreshed and harvested at 24 hrs intervals. All PCKS were harvested at 96 hrs.

### PCKS Harvest

Tissue culture supernatant (n=3 paired wells) were collected every 24 hrs and snap frozen for quantification of soluble outputs. At 96 hrs n=4 PCKS were snap frozen for RNA extraction and n=2 PCKS were formalin fixed paraffin embedded (FFPE) for IHC.

### PCKS Function and Viability Analysis

Tissue culture levels of markers of kidney damage (Lactate Dehydrogenase (LDH) and KIM-1) were quantified on all PCKS at all-time points (n=165 samples per donor (n=3 @ 24 hrs, n=54 @ 48hrs, 72 hrs, 96 hrs)).

### Soluble Outputs Analysis

Levels of collagen 1a1, IL-6, Hyaluronic Acid and TIMP-1 in the tissue culture supernatants (n=165 samples per donor) were quantified using R&D Duoset ELISA kits.

### Quantitative Real Time PCR

Total RNA extraction from PCKS was performed on all samples (n=19 samples per donor (including TO PCKS), 4 pooled PCKS per condition). For RNA extraction RNAeasy Mini kit (Qiagen) was used. RNA was reverse transcribed to cDNA and used in qPCRs to measure transcript levels of AXL, Gas6, Col1a1, αSMA, TIMP-1, TGF-β1, IL-6 and β-actin/GAPDH.

### Histological Analysis

FFPE tissue was cut into 5 μm sections and stained for Picrosirius Red and α-Smooth Muscle Actin (αSMA) (n=19 samples per donor). Analysis was performed by a pathology trained technical staff member without knowledge of the sample ID. High magnification images were taken of each slide and analysed for the surface area covered by the stain (e.g. percentage area positive for Oil Red O, Picrosirius or αSMA).

### RESULTS

Soluble outputs analysis

Levels of collagen 1a1 in the tissue culture supernatants were quantified using R&D Duoset ELISA kits. Selected results are shown in FIG. 1.

### Histological Analysis

α-SMA stained PCKS sections were prepared. Up to 25 high magnification images (×20) from each PCKS were quantified for the percentage area of tissue positive for stain and averaged to give a mean tissue area positive for α-SMA. Selected results are shown in FIGS. 2 & 3.

### Conclusions

### Summary

Human kidney PCKS were generated and treated with a range of inhibitors and biologics (Bemcentinib, Nintedanib, Selonsertib, Enalapril and Tilvestamab) at a range of doses and combinations. Tissue viability was not adversely affected by any of the client's drugs or other treatments. Although a few samples showed a minor increase in LDH at the start of the experiment, this was an expected after-effect of tissue slicing which completely settled as experiment progressed. KIM-1 appeared to be stable over the first 48 hours of experiment with a slow increase over the course of the experiment, which is in keeping with the responses of an aged kidney. No significant increase of KIM-1 was detected in any of the samples with various range of treatments.

### Soluble Markers

As would be predicted for this model, the positive controls Alk5i at 10 μM and Nintedanib at 4 μM inhibited Collagen 1a1 production at 7 2hrs and 96 hrs of treatment. Similar anti-fibrotic activity was seen in PCKS treated with Selonsertib.

In terms of efficacy of the AXLi agents to reduce fibrogenesis/fibrosis, it was found that Bemcentinib was able to reduce Collagen 1a1 production at all doses (albeit not in a dose dependent manner), while the treatment with Enalapril alone or in combination with Bemcentinib did not show any efficacy towards reducing Collagen 1a1 secretion. Treatment with Tilvestamab had no effect on reducing Collagen 1a1 secretion, however combination treatment of lowest dose Tilvestamab with low dose Enalapril (0.15 μM) had a profound effect on reducing Collagen 1a1 secretion, which was comparable to Alk5i and Nintedanib.

Levels of IL-6 in the media remained relatively unchanged, with the exception of a spike in IL-6 secretion at the early time point (48 hrs) in PCKS treated with two of the high doses of Bemcentinib.

Hyaluronic acid was readily reduced with Nintedanib, however other treatments had a very limited effect.

The positive controls Alk5 inhibitor at 10 μM and Nintedanib at 4 μM were able to significantly inhibit TIMP-1 production at 72 hrs and 96 hrs of treatment. Similarly, it was found that Bemcentinib was able to reduce TIMP-1 production at all doses, while the treatment with Enalapril alone or in combination with Bemcentinib did not show any efficacy towards reducing TIMP-1 secretion. Treatment with Tilvestamab had no effect on reducing TIMP-1 secretion, however combination treatment of lowest dose Tilvestamab with low dose Enalapril (0.15 μM) significantly reduced TIMP-1 secretion.

qPCR

Transcriptional activity of AXL and Gas6 showed some variation between the treatment groups, however the changes are confided to no more than 1.5-2 fold change in gene expression. Collagen 1a1 transcriptional activity broadly followed the pattern of Collagen 1a1 secretion as discussed previously.

### Histology

Histological effects of treatments were assessed on the basis of changes in percentage positive area stained for αSMA (activated myofibroblasts) and Picrosirius Red (collagen deposition).

αSMA was reduced in PCKS treated singly with 3 μM Bemcentinib or 0.15 μM Enalapril as well as three of the highest doses of Tilvestamab. Combination treatment of lowest dose Tilvestamab with low dose Enalapril (0.15 μM) had the greatest amount of reduction in αSMA, which was 50% lower than the control. These changes were not evident in Picrosirius Red staining, which appeared to be relatively stable.

### Example 2

### Materials & Methods

### PCKS Preparation and Culture

Precision cut kidney slices (PCKS) were prepared from explanted kidney tissue and rested for 24 hours to allow the post-slicing stress period to elapse before experiments were initiated. PCKS were then cultured without exogenous challenge in the presence or absence of ALK5i (10 μM) as a positive control or novel inhibitors at indicated concentrations. PCKS culture media, including all compounds, were refreshed and harvested at 24 hrs intervals. All PCKS were harvested at 96 hrs.

### Study Groups

PCKS were prepared from n=1 individual human kidney. The human kidneys are unused donor kidneys that are assessed but not utilised for transplant.

### Marker Analysis

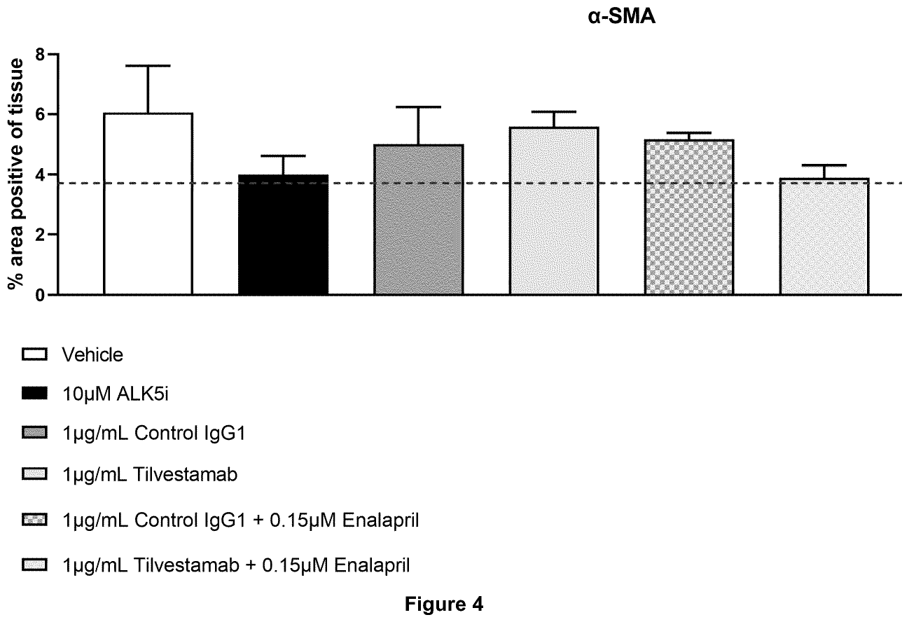

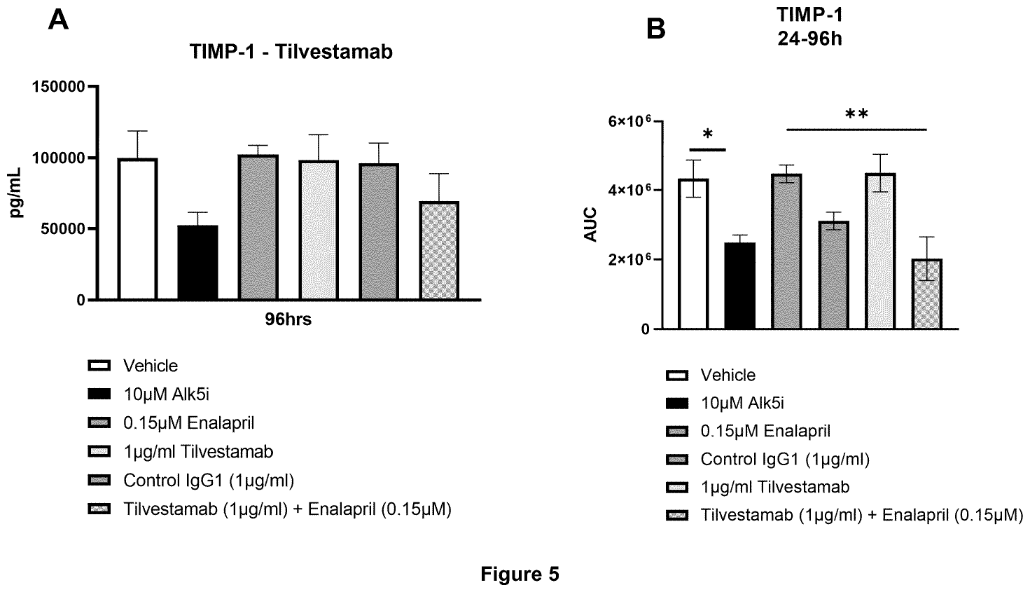

Levels of TIMP metallopeptidase inhibitor 1 were quantified in tissue culture supernatants using using R&D Duoset ELISA kits—see FIGS. 5A and 5B. Two PCKS were formalin fixed paraffin embedded (FFPE) for α-Smooth Muscle Actin (αSMA) staining—see FIG. 4.

### Conclusions

Results from the second donor tissue is consistent with those reported in Example 1, above, showing a synergistic effect between Tilvestamab and Enalapril in the suppression of fibrotic markers.

### REFERENCES

A number of publications are cited above in order to more fully describe and disclose the invention and the state of the art to which the invention pertains. Citations for these references are provided in the text. The entirety of each of these references is incorporated herein.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

1. An antibody that binds Axl and anatagonizes Axl activity for use in a method of treating a fibrotic disease, the method comprising administering to a subject in need thereof a therapeutically effective amount of the antibody in combination with a RAS inhibitor.

2. The antibody for use of claim 1, wherein the anti-AXL antibody inhibits Axl kinase activity, inhibits Axl signalling activity, inhibits the binding of AXL to the GAS6 ligand, and/or crosslinks Axl receptors.

3. The antibody for use of either of claim 1 or 2, wherein the anti-AXL antibody comprises the CDRs of the VH domain having the sequence of SEQ ID No. 1 and/or the CDRs of VL domain having the sequence set out herein as SEQ ID No.2;
optionally wherein the anti-AXL antibody comprises the 6 CDRs having the sequences set out herein as: SEQ ID Nos. 3 to 8, SEQ ID Nos. 9 to 14, SEQ ID Nos. 15 to 20, SEQ ID Nos. 21 to 26, or SEQ ID Nos. 27 to 32.

4. The antibody for use of claim 1, wherein the anti-AXL antibody is Tilvestamab.

5. The antibody for use of any one of claims 1 to 4, wherein the RAS inhibitor is an ACE inhibitor.

6. The antibody for use of claim 1, wherein the ACE inhibitor is Enalapril.

7. The antibody for use of any one of claims 1 to 4, wherein the RAS inhibitor is an ARB.

8. The antibody for use of any one of claims 1 to 4, wherein the RAS inhibitor is an AA.

9. The antibody for use of any one of claims 1 to 8, wherein the fibrotic disorder is selected from the grou consisting of: arthrofibrosis, Crohn's disease, Dupuytren's contracture, peyronie's disease, strabmisus, scleroderma, keloid, Nephrogenic systemic fibrosis, pulmonary fibrosis, idiopathic pulmonary fibrosis (IPF), cystic fibrosis (CF), progressive massive fibrosis, fibrothorax, systemic sclerosis, cardiac fibrosis, non-alcoholic steatohepatitis (NASH), other types of liver fibrosis, primary biliary cirrhosis, renal fibrosis, reteroperitoneal fibrosis, mediastinal fibrosis, myelofibrosis, and atherosclerosis.

10. The antibody for use of any one of claims 1 to 9, wherein the fibrotic disorder is kidney fibrosis.

11. The antibody for use of any one of claims 1 to 10, wherein the antibody is administered concurrently with the RAS inhibitor.

12. The antibody for use of any one of claims 1 to 11, wherein the antibody is administered separately and/or sequentially to the RAS inhibitor.

13. The antibody for use of any one of claims 1 to 12, wherein the method comprises administering the antibody to the subject, wherein the RAS inhibitor has been, is, or will be, administered to the subject.

14. A pharmaceutical composition comprising: an antibody as defined in any one of claims 1 to 4; a RAS inhibitor as defined in any one of claims 5 to 8; and, a pharmaceutically acceptable excipient.

15. A method of selecting a subject for treatment with an antibody as defined in any one of claims 1 to 4,
wherein a subject is selected for treatment if the subject has been, will be, or is being treated with a RAS inhibitor.

16. A method of treating a fibrotic disease, the method comprising administering to a subject in need thereof a therapeutically effective amount of an antibody that binds Axl and anatagonizes Axl activity in combination with a RAS inhibitor.

17. The method according to claim 16, wherein the antibody, RAS inhibitor, fibrotic disease, and/or one or more method steps are as defined in any one of claims 2 to 13.

18. Use of a therapeutically effective amount of an antibody that binds Axl and anatagonizes Axl activity for the manufacture of a medicament for treating a fibrotic disease in a subject in need thereof, wherein the medicament is admistered in combination with a RAS inhibitor.

19. Use of a combination of a therapeutically effective amount of an antibody that binds Axl and anatagonizes Axl activity and a RAS inhibitor for the manufacture of a medicament for treating a fibrotic disease in a subject in need thereof.

20. The use according to either one of claim 18 or 19, wherein the antibody, RAS inhibitor, fibrotic disease, and/or one or more method steps are as defined in any one of claims 2 to 13.


SEQUENCE LISTING (248 sequences)

SEQ ID NO: 1 - protein, 118 aa, Artificial Sequence
  BGB149 VH
EVQLVESGGGLVQPGGSLRLSCAASGYSFTDFYINWVRQAPGKGLEWVARIFPGGDNTYYNEKFKGRFTLSADTSKSTAYLQMNSLRAEDTAVYYCARRGLYYAMDYWGQGTLVTVSS

SEQ ID NO: 2 - protein, 112 aa, Artificial Sequence
  BGB149 VL
DIQMTQSPSSLSASVGDRVTITCRSSQSLVHSNGIPYLHWYQQKPGKAPKLLIYRVSNRFSGVPSRFSGSGSGTDFTLTISSLQPEDFATYYCSQGTHVPPTFGQGTKVEIK

SEQ ID NO: 3 - protein, 7 aa, Artificial Sequence
  BGB149 HCDR1 chothia definition
GYSFTDF

SEQ ID NO: 4 - protein, 6 aa, Artificial Sequence
  BGB149 HCDR2 chothia definition
FPGGDN

SEQ ID NO: 5 - protein, 9 aa, Artificial Sequence
  BGB149 HCDR3 chothia definition
RGLYYAMDY

SEQ ID NO: 6 - protein, 16 aa, Artificial Sequence
  BGB149 LCDR1 chothia definition
RSSQSLVHSNGIPYLH

SEQ ID NO: 7 - protein, 7 aa, Artificial Sequence
  BGB149 LCDR2 chothia definition
RVSNRFS

SEQ ID NO: 8 - protein, 9 aa, Artificial Sequence
  BGB149 LCDR3 chothia definition
SQGTHVPPT

SEQ ID NO: 9 - protein, 10 aa, Artificial Sequence
  BGB149 HCDR1 AbM definition
GYSFTDFYIN

SEQ ID NO: 10 - protein, 10 aa, Artificial Sequence
  BGB149 HCDR2 AbM definition
RIFPGGDNTY

SEQ ID NO: 11 - protein, 9 aa, Artificial Sequence
  BGB149 HCDR3 AbM definition
RGLYYAMDY

SEQ ID NO: 12 - protein, 16 aa, Artificial Sequence
  BGB149 LCDR1 AbM definition
RSSQSLVHSNGIPYLH

SEQ ID NO: 13 - protein, 7 aa, Artificial Sequence
  BGB149 LCDR2 AbM definition
RVSNRFS

SEQ ID NO: 14 - protein, 9 aa, Artificial Sequence
  BGB149 LCDR3 AbM definition
SQGTHVPPT

SEQ ID NO: 15 - protein, 5 aa, Artificial Sequence
  BGB149 HCDR1 Kabat definition
DFYIN

SEQ ID NO: 16 - protein, 17 aa, Artificial Sequence
  BGB149 HCDR2 Kabat definition
RIFPGGDNTYYNEKFKG

SEQ ID NO: 17 - protein, 9 aa, Artificial Sequence
  BGB149 HCDR3 Kabat definition
RGLYYAMDY

SEQ ID NO: 18 - protein, 16 aa, Artificial Sequence
  BGB149 LCDR1 Kabat definition
RSSQSLVHSNGIPYLH

SEQ ID NO: 19 - protein, 7 aa, Artificial Sequence
  BGB149 LCDR2 Kabat definition
RVSNRFS

SEQ ID NO: 20 - protein, 9 aa, Artificial Sequence
  BGB149 LCDR3 Kabat definition
SQGTHVPPT

SEQ ID NO: 21 - protein, 6 aa, Artificial Sequence
  BGB149 HCDR1 contact definition
TDFYIN

SEQ ID NO: 22 - protein, 13 aa, Artificial Sequence
  BGB149 HCDR2 contact definition
WVARIFPGGDNTY

SEQ ID NO: 23 - protein, 10 aa, Artificial Sequence
  BGB149 HCDR3 contact definition
ARRGLYYAMD

SEQ ID NO: 24 - protein, 12 aa, Artificial Sequence
  BGB149 LCDR1 contact definition
VHSNGIPYLHWY

SEQ ID NO: 25 - protein, 10 aa, Artificial Sequence
  BGB149 LCDR2 contact definition
LLIYRVSNRF

SEQ ID NO: 26 - protein, 8 aa, Artificial Sequence
  BGB149 LCDR3 contact definition
SQGTHVPP

SEQ ID NO: 27 - protein, 8 aa, Artificial Sequence
  BGB149 HCDR1 IMGT definition
GYSFTDFY

SEQ ID NO: 28 - protein, 8 aa, Artificial Sequence
  BGB149 HCDR2 IMGT definition
IFPGGDNT

SEQ ID NO: 29 - protein, 11 aa, Artificial Sequence
  BGB149 HCDR3 IMGT definition
ARRGLYYAMDY

SEQ ID NO: 30 - protein, 11 aa, Artificial Sequence
  BGB149 LCDR1 IMGT definition
QSLVHSNGIPY

SEQ ID NO: 31 - protein, 9 aa, Artificial Sequence
  BGB149 LCDR3 IMGT definition
SQGTHVPPT

SEQ ID NO: 32 - protein, 118 aa, Artificial Sequence
  10G5alt VH
EVQLVQSGAGLVQPGGSVRLSCAASGYSFTDFYINWVRQAPGKGLEWIARIFPGGDNTYYNEKFKGRFTLSADTSSSTAYLQLNSLRAEDTAVYYCARRGLYYAMDYWGQGTLVTVSS

SEQ ID NO: 33 - protein, 112 aa, Artificial Sequence
  10G5alt VL
DIQMTQSPSSLSASVGDRVTITCRSSQSLVHSNGIPYLHWYQQKPGKAPKLLIYRVSNRFSGVPSRFSGSRSGTDFTLTISSLQPEDFATYYCSQGTHVPPTFGQGTKVEIK

SEQ ID NO: 34 - protein, 7 aa, Artificial Sequence
  10G5alt HCDR1 chothia definition
GYSFTDF

SEQ ID NO: 35 - protein, 6 aa, Artificial Sequence
  10G5alt HCDR2 chothia definition
FPGGDN

SEQ ID NO: 36 - protein, 9 aa, Artificial Sequence
  10G5alt HCDR3 chothia definition
RGLYYAMDY

SEQ ID NO: 37 - protein, 16 aa, Artificial Sequence
  10G5alt LCDR1 chothia definition
RSSQSLVHSNGIPYLH

SEQ ID NO: 38 - protein, 7 aa, Artificial Sequence
  10G5alt LCDR2 chothia definition
RVSNRFS

SEQ ID NO: 39 - protein, 9 aa, Artificial Sequence
  10G5alt LCDR3 chothia definition
SQGTHVPPT

SEQ ID NO: 40 - protein, 10 aa, Artificial Sequence
  10G5alt HCDR1 AbM definition
GYSFTDFYIN

SEQ ID NO: 41 - protein, 10 aa, Artificial Sequence
  10G5alt HCDR2 AbM definition
RIFPGGDNTY

SEQ ID NO: 42 - protein, 9 aa, Artificial Sequence
  10G5alt HCDR3 AbM definition
RGLYYAMDY

SEQ ID NO: 43 - protein, 16 aa, Artificial Sequence
  10G5alt LCDR1 AbM definition
RSSQSLVHSNGIPYLH

SEQ ID NO: 44 - protein, 7 aa, Artificial Sequence
  10G5alt LCDR2 AbM definition
RVSNRFS

SEQ ID NO: 45 - protein, 9 aa, Artificial Sequence
  10G5alt LCDR3 AbM definition
SQGTHVPPT

SEQ ID NO: 46 - protein, 5 aa, Artificial Sequence
  10G5alt HCDR1 Kabat definition
DFYIN

SEQ ID NO: 47 - protein, 17 aa, Artificial Sequence
  10G5alt HCDR2 Kabat definition
RIFPGGDNTYYNEKFKG

SEQ ID NO: 48 - protein, 9 aa, Artificial Sequence
  10G5alt HCDR3 Kabat definition
RGLYYAMDY

SEQ ID NO: 49 - protein, 16 aa, Artificial Sequence
  10G5alt LCDR1 Kabat definition
RSSQSLVHSNGIPYLH

SEQ ID NO: 50 - protein, 7 aa, Artificial Sequence
  10G5alt LCDR2 Kabat definition
RVSNRFS

SEQ ID NO: 51 - protein, 9 aa, Artificial Sequence
  10G5alt LCDR3 Kabat definition
SQGTHVPPT

SEQ ID NO: 52 - protein, 6 aa, Artificial Sequence
  10G5alt HCDR1 Contact definition
TDFYIN

SEQ ID NO: 53 - protein, 13 aa, Artificial Sequence
  10G5alt HCDR2 Contact definition
WIARIFPGGDNTY

SEQ ID NO: 54 - protein, 10 aa, Artificial Sequence
  10G5alt HCDR3 Contact definition
ARRGLYYAMD

SEQ ID NO: 55 - protein, 12 aa, Artificial Sequence
  10G5alt LCDR1 Contact definition
VHSNGIPYLHWY

SEQ ID NO: 56 - protein, 10 aa, Artificial Sequence
  10G5alt LCDR2 Contact definition
LLIYRVSNRF

SEQ ID NO: 57 - protein, 8 aa, Artificial Sequence
  10G5alt LCDR3 Contact definition
SQGTHVPP

SEQ ID NO: 58 - protein, 8 aa, Artificial Sequence
  10G5alt HCDR1 IMGT definition
GYSFTDFY

SEQ ID NO: 59 - protein, 8 aa, Artificial Sequence
  10G5alt HCDR2 IMGT definition
IFPGGDNT

SEQ ID NO: 60 - protein, 11 aa, Artificial Sequence
  10G5alt HCDR3 IMGT definition
ARRGLYYAMDY

SEQ ID NO: 61 - protein, 11 aa, Artificial Sequence
  10G5alt LCDR1 IMGT definition
QSLVHSNGIPY

SEQ ID NO: 62 - protein, 9 aa, Artificial Sequence
  10G5alt LCDR3 IMGT definition
SQGTHVPPT

SEQ ID NO: 63 - protein, 118 aa, Artificial Sequence
  m10C9 VH
QVQLQQPGPELVKPGASVKISCKTSDYNFTRYYIHWVKQRPGQGLEWIGWIYPGTGDSKYNEKFKGRATLTADTSSSTAYMQLSSQTSEDSAVYFCARNGNYWYFDVWGAGTAVTVSS

SEQ ID NO: 64 - protein, 112 aa, Artificial Sequence
  m10C9 VL
DIVMTQAAPSGPVTPGESVSISCRSSKSLLHSNGNTYLYWFLQRPGQSPQLLIYRMSNLASGVPDRFSGSGSGTAFTLRISRVEAEDVGIYYCMQHREYPFTFGGGTKLEIK

SEQ ID NO: 65 - protein, 7 aa, Artificial Sequence
  m10C9 HCDR1 Chothia definition
DYNFTRY

SEQ ID NO: 66 - protein, 6 aa, Artificial Sequence
  m10C9 HCDR2 Chothia definition
YPGTGD

SEQ ID NO: 67 - protein, 9 aa, Artificial Sequence
  m10C9 HCDR3 Chothia definition
NGNYWYFDV

SEQ ID NO: 68 - protein, 16 aa, Artificial Sequence
  m10C9 LCDR1 Chothia definition
RSSKSLLHSNGNTYLY

SEQ ID NO: 69 - protein, 7 aa, Artificial Sequence
  m10C9 LCDR2  Chothia definition
RMSNLAS

SEQ ID NO: 70 - protein, 9 aa, Artificial Sequence
  m10C9 LCDR3 Chothia definition
MQHREYPFT

SEQ ID NO: 71 - protein, 10 aa, Artificial Sequence
  m10C9 HCDR1 AbM definition
DYNFTRYYIH

SEQ ID NO: 72 - protein, 10 aa, Artificial Sequence
  m10C9 HCDR2 AbM definition
WIYPGTGDSK

SEQ ID NO: 73 - protein, 9 aa, Artificial Sequence
  m10C9 HCDR3 AbM definition
NGNYWYFDV

SEQ ID NO: 74 - protein, 16 aa, Artificial Sequence
  m10C9 LCDR1 AbM definition
RSSKSLLHSNGNTYLY

SEQ ID NO: 75 - protein, 7 aa, Artificial Sequence
  m10C9 LCDR2 AbM definition
RMSNLAS

SEQ ID NO: 76 - protein, 9 aa, Artificial Sequence
  m10C9 LCDR3 AbM definition
MQHREYPFT

SEQ ID NO: 77 - protein, 5 aa, Artificial Sequence
  m10C9 HCDR1 Kabat definition
RYYIH

SEQ ID NO: 78 - protein, 17 aa, Artificial Sequence
  m10C9 HCDR2 Kabat definition
WIYPGTGDSKYNEKFKG

SEQ ID NO: 79 - protein, 9 aa, Artificial Sequence
  m10C9 HCDR3 Kabat definition
NGNYWYFDV

SEQ ID NO: 80 - protein, 16 aa, Artificial Sequence
  m10C9 LCDR1 Kabat definition
RSSKSLLHSNGNTYLY

SEQ ID NO: 81 - protein, 7 aa, Artificial Sequence
  m10C9 LCDR2 Kabat definition
RMSNLAS

SEQ ID NO: 82 - protein, 9 aa, Artificial Sequence
  m10C9 LCDR3 Kabat definition
MQHREYPFT

SEQ ID NO: 83 - protein, 6 aa, Artificial Sequence
  m10C9 HCDR1 Contact definition
TRYYIH

SEQ ID NO: 84 - protein, 13 aa, Artificial Sequence
  m10C9 HCDR2 Contact definition
WIGWIYPGTGDSK

SEQ ID NO: 85 - protein, 10 aa, Artificial Sequence
  m10C9 HCDR3 Contact definition
ARNGNYWYFD

SEQ ID NO: 86 - protein, 12 aa, Artificial Sequence
  m10C9 LCDR1 Contact definition
LHSNGNTYLYWF

SEQ ID NO: 87 - protein, 10 aa, Artificial Sequence
  m10C9 LCDR2 Contact definition
LLIYRMSNLA

SEQ ID NO: 88 - protein, 8 aa, Artificial Sequence
  m10C9 LCDR3 Contact definition
MQHREYPF

SEQ ID NO: 89 - protein, 8 aa, Artificial Sequence
  m10C9 HCDR1 IMGT definition
DYNFTRYY

SEQ ID NO: 90 - protein, 8 aa, Artificial Sequence
  m10C9 HCDR2 IMGT definition
IYPGTGDS

SEQ ID NO: 91 - protein, 11 aa, Artificial Sequence
  m10C9 HCDR3 IMGT definition
ARNGNYWYFDV

SEQ ID NO: 92 - protein, 11 aa, Artificial Sequence
  m10C9 LCDR1 IMGT definition
KSLLHSNGNTY

SEQ ID NO: 93 - protein, 9 aa, Artificial Sequence
  m10C9 LCDR3 IMGT definition
MQHREYPFT

SEQ ID NO: 94 - protein, 125 aa, Artificial Sequence
  YW327.6S2 VH
EVQLVESGGGLVQPGGSLRLSCAASGFSLSGSWIHWVRQAPGKGLEWVGWINPYRGYAYYADSVKGRFTISADTSKNTAYLQMNSLRAEDTAVYYCAREYSGWGGSSVGYAMDYWGQGTLVTVSS

SEQ ID NO: 95 - protein, 108 aa, Artificial Sequence
  YW327.6S2 VL
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 96 - protein, 7 aa, Artificial Sequence
  YW327.6S2 HCDR1 Chothia definition
GFSLSGS

SEQ ID NO: 97 - protein, 6 aa, Artificial Sequence
  YW327.6S2 HCDR2 Chothia definition
NPYRGY

SEQ ID NO: 98 - protein, 16 aa, Artificial Sequence
  YW327.6S2 HCDR3 Chothia definition
EYSGWGGSSVGYAMDY

SEQ ID NO: 99 - protein, 11 aa, Artificial Sequence
  YW327.6S2 LCDR1 Chothia definition
RASQDVSTAVA

SEQ ID NO: 100 - protein, 7 aa, Artificial Sequence
  YW327.6S2 LCDR2 Chothia definition
SASFLYS

SEQ ID NO: 101 - protein, 9 aa, Artificial Sequence
  YW327.6S2 LCDR3 Chothia definition
QQSYTTPPT

SEQ ID NO: 102 - protein, 10 aa, Artificial Sequence
  YW327.6S2 HCDR1 AbM definition
GFSLSGSWIH

SEQ ID NO: 103 - protein, 10 aa, Artificial Sequence
  YW327.6S2 HCDR2 AbM definition
WINPYRGYAY

SEQ ID NO: 104 - protein, 16 aa, Artificial Sequence
  YW327.6S2 HCDR3 AbM definition
EYSGWGGSSVGYAMDY

SEQ ID NO: 105 - protein, 11 aa, Artificial Sequence
  YW327.6S2 LCDR1 AbM definition
RASQDVSTAVA

SEQ ID NO: 106 - protein, 7 aa, Artificial Sequence
  YW327.6S2 LCDR2 AbM definition
SASFLYS

SEQ ID NO: 107 - protein, 9 aa, Artificial Sequence
  YW327.6S2 LCDR3 AbM definition
QQSYTTPPT

SEQ ID NO: 108 - protein, 5 aa, Artificial Sequence
  YW327.6S2 HCDR1 Kabat definition
GSWIH

SEQ ID NO: 109 - protein, 17 aa, Artificial Sequence
  YW327.6S2 HCDR2 Kabat definition
WINPYRGYAYYADSVKG

SEQ ID NO: 110 - protein, 16 aa, Artificial Sequence
  YW327.6S2 HCDR3 Kabat definition
EYSGWGGSSVGYAMDY

SEQ ID NO: 111 - protein, 11 aa, Artificial Sequence
  YW327.6S2 LCDR1 Kabat definition
RASQDVSTAVA

SEQ ID NO: 112 - protein, 7 aa, Artificial Sequence
  YW327.6S2 LCDR2 Kabat definition
SASFLYS

SEQ ID NO: 113 - protein, 9 aa, Artificial Sequence
  YW327.6S2 LCDR3 Kabat definition
QQSYTTPPT

SEQ ID NO: 114 - protein, 6 aa, Artificial Sequence
  YW327.6S2 HCDR1 Contact definition
SGSWIH

SEQ ID NO: 115 - protein, 13 aa, Artificial Sequence
  YW327.6S2 HCDR2 Contact definition
WVGWINPYRGYAY

SEQ ID NO: 116 - protein, 17 aa, Artificial Sequence
  YW327.6S2 HCDR3 Contact definition
AREYSGWGGSSVGYAMD

SEQ ID NO: 117 - protein, 7 aa, Artificial Sequence
  YW327.6S2 LCDR1 Contact definition
STAVAWY

SEQ ID NO: 118 - protein, 10 aa, Artificial Sequence
  YW327.6S2 LCDR2 Contact definition
LLIYSASFLY

SEQ ID NO: 119 - protein, 8 aa, Artificial Sequence
  YW327.6S2 LCDR3 Contact definition
QQSYTTPP

SEQ ID NO: 120 - protein, 8 aa, Artificial Sequence
  YW327.6S2 HCDR1 IMGT definition
GFSLSGSW

SEQ ID NO: 121 - protein, 8 aa, Artificial Sequence
  YW327.6S2 HCDR2 IMGT definition
INPYRGYA

SEQ ID NO: 122 - protein, 18 aa, Artificial Sequence
  YW327.6S2 HCDR3 IMGT definition
AREYSGWGGSSVGYAMDY

SEQ ID NO: 123 - protein, 6 aa, Artificial Sequence
  YW327.6S2 LCDR1 IMGT definition
QDVSTA

SEQ ID NO: 124 - protein, 9 aa, Artificial Sequence
  YW327.6S2 LCDR3 IMGT definition
QQSYTTPPT

SEQ ID NO: 125 - protein, 120 aa, Artificial Sequence
  1003A2 VH
EVKLVESGGGLVQPGGSLKLSCAASGFTFNSYSMSWVRQTPEKRLEWVAYISNGGGRTYYPDTIKGRFTLSRDNDRNTLYLQMSSLKSEDTAMYYCARGTTESYYALDYWGQGTSVTVSS

SEQ ID NO: 126 - protein, 107 aa, Artificial Sequence
  1003A2 VL
DIVMTQSHKFMSTSLGDRISITCKASQDVITAVAWYQQKPGQSPKLLIYWASTRHTGVPDRFTGSGSGTDYNLTISSVQAEDLALYYCQQHFSTPLTFGAGTKLELK

SEQ ID NO: 127 - protein, 7 aa, Artificial Sequence
  1003A2 HCDR1 Chothia definition
GFTFNSY

SEQ ID NO: 128 - protein, 6 aa, Artificial Sequence
  1003A2 HCDR2 Chothia definition
SNGGGR

SEQ ID NO: 129 - protein, 11 aa, Artificial Sequence
  1003A2 HCDR3 Chothia definition
GTTESYYALDY

SEQ ID NO: 130 - protein, 11 aa, Artificial Sequence
  1003A2 LCDR1 Chothia definition
KASQDVITAVA

SEQ ID NO: 131 - protein, 7 aa, Artificial Sequence
  1003A2 LCDR2 Chothia definition
WASTRHT

SEQ ID NO: 132 - protein, 9 aa, Artificial Sequence
  1003A2 LCDR3 Chothia definition
QQHFSTPLT

SEQ ID NO: 133 - protein, 10 aa, Artificial Sequence
  1003A2 HCDR1 AbM definition
GFTFNSYSMS

SEQ ID NO: 134 - protein, 10 aa, Artificial Sequence
  1003A2 HCDR2 AbM definition
YISNGGGRTY

SEQ ID NO: 135 - protein, 11 aa, Artificial Sequence
  1003A2 HCDR3 AbM definition
GTTESYYALDY

SEQ ID NO: 136 - protein, 11 aa, Artificial Sequence
  1003A2 LCDR1 AbM definition
KASQDVITAVA

SEQ ID NO: 137 - protein, 7 aa, Artificial Sequence
  1003A2 LCDR2 AbM definition
WASTRHT

SEQ ID NO: 138 - protein, 9 aa, Artificial Sequence
  1003A2 LCDR3 AbM definition
QQHFSTPLT

SEQ ID NO: 139 - protein, 5 aa, Artificial Sequence
  1003A2 HCDR1 Kabat definition
SYSMS

SEQ ID NO: 140 - protein, 17 aa, Artificial Sequence
  1003A2 HCDR2 Kabat definition
YISNGGGRTYYPDTIKG

SEQ ID NO: 141 - protein, 11 aa, Artificial Sequence
  1003A2 HCDR3 Kabat definition
GTTESYYALDY

SEQ ID NO: 142 - protein, 11 aa, Artificial Sequence
  1003A2 LCDR1 Kabat definition
KASQDVITAVA

SEQ ID NO: 143 - protein, 7 aa, Artificial Sequence
  1003A2 LCDR2 Kabat definition
WASTRHT

SEQ ID NO: 144 - protein, 9 aa, Artificial Sequence
  1003A2 LCDR3 Kabat definition
QQHFSTPLT

SEQ ID NO: 145 - protein, 6 aa, Artificial Sequence
  1003A2 HCDR1 Contact definition
NSYSMS

SEQ ID NO: 146 - protein, 13 aa, Artificial Sequence
  1003A2 HCDR2 Contact definition
WVAYISNGGGRTY

SEQ ID NO: 147 - protein, 12 aa, Artificial Sequence
  1003A2 HCDR3 Contact definition
ARGTTESYYALD

SEQ ID NO: 148 - protein, 7 aa, Artificial Sequence
  1003A2 LCDR1 Contact definition
ITAVAWY

SEQ ID NO: 149 - protein, 10 aa, Artificial Sequence
  1003A2 LCDR1 Contact definition
LLIYWASTRH

SEQ ID NO: 150 - protein, 8 aa, Artificial Sequence
  1003A2 LCDR3 Contact definition
QQHFSTPL

SEQ ID NO: 151 - protein, 8 aa, Artificial Sequence
  1003A2 HCDR1 IMGT definition
GFTFNSYS

SEQ ID NO: 152 - protein, 8 aa, Artificial Sequence
  1003A2 HCDR2 IMGT definition
ISNGGGRT

SEQ ID NO: 153 - protein, 13 aa, Artificial Sequence
  1003A2 HCDR3 IMGT definition
ARGTTESYYALDY

SEQ ID NO: 154 - protein, 6 aa, Artificial Sequence
  1003A2 LCDR1 IMGT definition
QDVITA

SEQ ID NO: 155 - protein, 9 aa, Artificial Sequence
  1003A2 LCDR3 IMGT definition
QQHFSTPLT

SEQ ID NO: 156 - protein, 124 aa, Artificial Sequence
  1613F12 VH
EVHLQQSGAELVKPGASVKLSCTASGFNIRDTYIHWVKQRPEQGLEWIGRLDPANGHTKYGPNFQGRATMTSDTSSNTAYLQLSSLTSEDTAVYYCARGAYYYGSSGLFYFDYWGQGTTLSVSS

SEQ ID NO: 157 - protein, 107 aa, Artificial Sequence
  1613F12 VL
DVQITQSPSYLATSPGETITINCRASKSISKYLAWYQEKPGKTNKLLIYSGSTLQSGVPSRFSGSGSGTDFTLTISSLEPEDFAMYFCQQHHEYPLTFGAGTELELK

SEQ ID NO: 158 - protein, 7 aa, Artificial Sequence
  1613F12 HCDR1 Chothia definition
GFNIRDT

SEQ ID NO: 159 - protein, 6 aa, Artificial Sequence
  1613F12 HCDR2 Chothia definition
DPANGH

SEQ ID NO: 160 - protein, 15 aa, Artificial Sequence
  1613F12 HCDR3 Chothia definition
GAYYYGSSGLFYFDY

SEQ ID NO: 161 - protein, 11 aa, Artificial Sequence
  1613F12 LCDR1 Chothia definition
RASKSISKYLA

SEQ ID NO: 162 - protein, 7 aa, Artificial Sequence
  1613F12 LCDR2 Chothia definition
SGSTLQS

SEQ ID NO: 163 - protein, 9 aa, Artificial Sequence
  1613F12 LCDR3 Chothia definition
QQHHEYPLT

SEQ ID NO: 164 - protein, 10 aa, Artificial Sequence
  1613F12 HCDR1 AbM definition
GFNIRDTYIH

SEQ ID NO: 165 - protein, 10 aa, Artificial Sequence
  1613F12 HCDR2 AbM definition
RLDPANGHTK

SEQ ID NO: 166 - protein, 15 aa, Artificial Sequence
  1613F12 HCDR3 AbM definition
GAYYYGSSGLFYFDY

SEQ ID NO: 167 - protein, 11 aa, Artificial Sequence
  1613F12 LCDR1 AbM definition
RASKSISKYLA

SEQ ID NO: 168 - protein, 7 aa, Artificial Sequence
  1613F12 LCDR2 AbM definition
SGSTLQS

SEQ ID NO: 169 - protein, 9 aa, Artificial Sequence
  1613F12 LCDR3 AbM definition
QQHHEYPLT

SEQ ID NO: 170 - protein, 5 aa, Artificial Sequence
  1613F12 HCDR1 Kabat definition
DTYIH

SEQ ID NO: 171 - protein, 17 aa, Artificial Sequence
  1613F12 HCDR2 Kabat definition
RLDPANGHTKYGPNFQG

SEQ ID NO: 172 - protein, 15 aa, Artificial Sequence
  1613F12 HCDR3 Kabat definition
GAYYYGSSGLFYFDY

SEQ ID NO: 173 - protein, 11 aa, Artificial Sequence
  1613F12 LCDR1 Kabat definition
RASKSISKYLA

SEQ ID NO: 174 - protein, 7 aa, Artificial Sequence
  1613F12 LCDR2 Kabat definition
SGSTLQS

SEQ ID NO: 175 - protein, 9 aa, Artificial Sequence
  1613F12 LCDR3 Kabat definition
QQHHEYPLT

SEQ ID NO: 176 - protein, 6 aa, Artificial Sequence
  1613F12 HCDR1 Contact definition
RDTYIH

SEQ ID NO: 177 - protein, 13 aa, Artificial Sequence
  1613F12 HCDR2 Contact definition
WIGRLDPANGHTK

SEQ ID NO: 178 - protein, 16 aa, Artificial Sequence
  1613F12 HCDR3 Contact definition
ARGAYYYGSSGLFYFD

SEQ ID NO: 179 - protein, 7 aa, Artificial Sequence
  1613F12 HCDR3 Contact definition
SKYLAWY

SEQ ID NO: 180 - protein, 10 aa, Artificial Sequence
  1613F12 LCDR2 Contact definition
LLIYSGSTLQ

SEQ ID NO: 181 - protein, 8 aa, Artificial Sequence
  1613F12 LCDR3 Contact definition
QQHHEYPL

SEQ ID NO: 182 - protein, 8 aa, Artificial Sequence
  1613F12 HCDR1 IMGT definition
QQHHEYPL

SEQ ID NO: 183 - protein, 8 aa, Artificial Sequence
  1613F12 HCDR2 IMGT definition
LDPANGHT

SEQ ID NO: 184 - protein, 17 aa, Artificial Sequence
  1613F12 HCDR3 IMGT definition
ARGAYYYGSSGLFYFDY

SEQ ID NO: 185 - protein, 6 aa, Artificial Sequence
  1613F12 LCDR1 IMGT definition
KSISKY

SEQ ID NO: 186 - protein, 9 aa, Artificial Sequence
  1613F12 LCDR3 IMGT definition
QQHHEYPLT

SEQ ID NO: 187 - protein, 117 aa, Artificial Sequence
  110D7 VH
EVKLVESGGGLVQPGGSLKLSCATSGFTFSDYYMYWVRQTPEKRLEWVAYISNGGGSTYYPDTVKGRFTMSRDNAKNTLYLQMSRLKSEDTAMYYCARLSFYAMDYWGQGTSVTVSS

SEQ ID NO: 188 - protein, 107 aa, Artificial Sequence
  110D7 VH
DIVMTQSHKFMSTSVGDRVSITCKASQDVTTAVAWYQQKPGQSPKLLIYWASTRHTGVPDRFTGSGSGTDYTLTISSVQAEDLALYYCQQHYRIPFTFGSGTKLEIK

SEQ ID NO: 189 - protein, 107 aa, Artificial Sequence; identical to SEQ ID NO: 188
  110D7 HCDR1 Chothia definition

SEQ ID NO: 190 - protein, 6 aa, Artificial Sequence
  110D7 HCDR2 Chothia definition
SNGGGS

SEQ ID NO: 191 - protein, 8 aa, Artificial Sequence
  110D7 HCDR3 Chothia definition
LSFYAMDY

SEQ ID NO: 192 - protein, 11 aa, Artificial Sequence
  110D7 LCDR1 Chothia definition
KASQDVTTAVA

SEQ ID NO: 193 - protein, 7 aa, Artificial Sequence
  110D7 LCDR2 Chothia definition
WASTRHT

SEQ ID NO: 194 - protein, 9 aa, Artificial Sequence
  110D7 LCDR3 Chothia definition
QQHYRIPFT

SEQ ID NO: 195 - protein, 10 aa, Artificial Sequence
  110D7 HCDR1 AbM definition
GFTFSDYYMY

SEQ ID NO: 196 - protein, 10 aa, Artificial Sequence
  110D7 HCDR2 AbM definition
YISNGGGSTY

SEQ ID NO: 197 - protein, 8 aa, Artificial Sequence
  110D7 HCDR3 AbM definition
LSFYAMDY

SEQ ID NO: 198 - protein, 11 aa, Artificial Sequence
  110D7 LCDR1 AbM definition
KASQDVTTAVA

SEQ ID NO: 199 - protein, 7 aa, Artificial Sequence
  110D7 LCDR2 AbM definition
WASTRHT

SEQ ID NO: 200 - protein, 9 aa, Artificial Sequence
  110D7 LCDR3 AbM definition
QQHYRIPFT

SEQ ID NO: 201 - protein, 5 aa, Artificial Sequence
  110D7 HCDR1 Kabat definition
DYYMY

SEQ ID NO: 202 - protein, 17 aa, Artificial Sequence
  110D7 HCDR2 Kabat definition
YISNGGGSTYYPDTVKG

SEQ ID NO: 203 - protein, 8 aa, Artificial Sequence
  110D7 HCDR3 Kabat definition
LSFYAMDY

SEQ ID NO: 204 - protein, 11 aa, Artificial Sequence
  110D7 LCDR1 Kabat definition
KASQDVTTAVA

SEQ ID NO: 205 - protein, 7 aa, Artificial Sequence
  110D7 LCDR2 Kabat definition
WASTRHT

SEQ ID NO: 206 - protein, 9 aa, Artificial Sequence
  110D7 LCDR3 Kabat definition
QQHYRIPFT

SEQ ID NO: 207 - protein, 6 aa, Artificial Sequence
  110D7 HCDR1 Contact definition
SDYYMY

SEQ ID NO: 208 - protein, 13 aa, Artificial Sequence
  110D7 HCDR2 Contact definition
WVAYISNGGGSTY

SEQ ID NO: 209 - protein, 9 aa, Artificial Sequence
  110D7 HCDR3 Contact definition
ARLSFYAMD

SEQ ID NO: 210 - protein, 7 aa, Artificial Sequence
  110D7 LCDR1 Contact definition
TTAVAWY

SEQ ID NO: 211 - protein, 10 aa, Artificial Sequence
  110D7 LCDR2 Contact definition
LLIYWASTRH

SEQ ID NO: 212 - protein, 8 aa, Artificial Sequence
  110D7 LCDR3 Contact definition
QQHYRIPF

SEQ ID NO: 213 - protein, 8 aa, Artificial Sequence
  110D7 HCDR1 IMGT definition
GFTFSDYY

SEQ ID NO: 214 - protein, 8 aa, Artificial Sequence
  110D7 HCDR2 IMGT definition
ISNGGGST

SEQ ID NO: 215 - protein, 10 aa, Artificial Sequence
  110D7 HCDR3 IMGT definition
ARLSFYAMDY

SEQ ID NO: 216 - protein, 6 aa, Artificial Sequence
  110D7 LCDR1 IMGT definition
QDVTTA

SEQ ID NO: 217 - protein, 9 aa, Artificial Sequence
  110D7 LCDR3 IMGT definition
QQHYRIPFT

SEQ ID NO: 218 - protein, 120 aa, Artificial Sequence
  1024G11 VH
EVKLVESGGGLVQPGGSLNLSCAASGFTFSSYSMSWVRQTPEKRLEWVAFISNGGGRTYYPDTVKGRFTFSRDNARNTLYLQMSSLNSEDTAMYFCARGTTESYYAMDYWGQGTSVTVSS

SEQ ID NO: 219 - protein, 107 aa, Artificial Sequence
  1024G11 VL
DIVMTQSHKFMSTSVGDRVSITCKASQDVITAVAWYQQKPGQSPKLLIYWASTRHTGVPDRFTGSGSGTDYTLTISSVQAEDLALYFCQQHLSTPLTFGAGTKLELK

SEQ ID NO: 220 - protein, 7 aa, Artificial Sequence
  1024G11 HCDR1 Chothia definition
GFTFSSY

SEQ ID NO: 221 - protein, 6 aa, Artificial Sequence
  1024G11 HCDR2 Chothia definition
SNGGGR

SEQ ID NO: 222 - protein, 11 aa, Artificial Sequence
  1024G11 HCDR3 Chothia definition
GTTESYYAMDY

SEQ ID NO: 223 - protein, 11 aa, Artificial Sequence
  1024G11 LCDR1 Chothia definition
KASQDVITAVA

SEQ ID NO: 224 - protein, 7 aa, Artificial Sequence
  1024G11 LCDR2 Chothia definition
WASTRHT

SEQ ID NO: 225 - protein, 9 aa, Artificial Sequence
  1024G11 LCDR3 Chothia definition
QQHLSTPLT

SEQ ID NO: 226 - protein, 10 aa, Artificial Sequence
  1024G11 HCDR1 AbM definition
GFTFSSYSMS

SEQ ID NO: 227 - protein, 10 aa, Artificial Sequence
  1024G11 HCDR2 AbM definition
FISNGGGRTY

SEQ ID NO: 228 - protein, 11 aa, Artificial Sequence
  1024G11 HCDR3 AbM definition
GTTESYYAMDY

SEQ ID NO: 229 - protein, 11 aa, Artificial Sequence
  1024G11 LCDR1 AbM definition
KASQDVITAVA

SEQ ID NO: 230 - protein, 7 aa, Artificial Sequence
  1024G11 LCDR1 AbM definition
WASTRHT

SEQ ID NO: 231 - protein, 9 aa, Artificial Sequence
  1024G11 LCDR3 AbM definition
QQHLSTPLT

SEQ ID NO: 232 - protein, 5 aa, Artificial Sequence
  1024G11 HCDR1 Kabat definition
SYSMS

SEQ ID NO: 233 - protein, 17 aa, Artificial Sequence
  1024G11 HCDR2 Kabat definition
FISNGGGRTYYPDTVKG

SEQ ID NO: 234 - protein, 11 aa, Artificial Sequence
  1024G11 HCDR3 Kabat definition
GTTESYYAMDY

SEQ ID NO: 235 - protein, 11 aa, Artificial Sequence
  1024G11 LCDR1 Kabat definition
KASQDVITAVA

SEQ ID NO: 236 - protein, 7 aa, Artificial Sequence
  1024G11 LCDR2 Kabat definition
WASTRHT

SEQ ID NO: 237 - protein, 9 aa, Artificial Sequence
  1024G11 LCDR3 Kabat definition
QQHLSTPLT

SEQ ID NO: 238 - protein, 6 aa, Artificial Sequence
  1024G11 HCDR1 Contact definition
SSYSMS

SEQ ID NO: 239 - protein, 13 aa, Artificial Sequence
  1024G11 HCDR2 Contact definition
WVAFISNGGGRTY

SEQ ID NO: 240 - protein, 12 aa, Artificial Sequence
  1024G11 HCDR3 Contact definition
ARGTTESYYAMD

SEQ ID NO: 241 - protein, 7 aa, Artificial Sequence
  1024G11 LCDR1 Contact definition
ITAVAWY

SEQ ID NO: 242 - protein, 10 aa, Artificial Sequence
  1024G11 LCDR2 Contact definition
LLIYWASTRH

SEQ ID NO: 243 - protein, 8 aa, Artificial Sequence
  1024G11 LCDR3 Contact definition
QQHLSTPL

SEQ ID NO: 244 - protein, 8 aa, Artificial Sequence
  1024G11 HCDR1 IMGT definition
GFTFSSYS

SEQ ID NO: 245 - protein, 8 aa, Artificial Sequence
  1024G11 HCDR2 IMGT definition
ISNGGGRT

SEQ ID NO: 246 - protein, 13 aa, Artificial Sequence
  1024G11 HCDR3 IMGT definition
ARGTTESYYAMDY

SEQ ID NO: 247 - protein, 6 aa, Artificial Sequence
  1024G11 LCDR1 IMGT definition
QDVITA

SEQ ID NO: 248 - protein, 9 aa, Artificial Sequence
  1024G11 LCDR2 IMGT definition
QQHLSTPLT